(19) United States
(12) Patent Application Publication
(10) Pub. No.: US 2024/0010727 A1
(43) Pub. Date: Jan. 11, 2024

(54) COMPOSITIONS AND METHODS FOR TREATING CANCER WITH A COMBINATION OF AN ANTAGONIST OF PD-1 AND AN ANTI-CTLA4 ANTIBODY

(71) Applicant: Merck Sharp & Dohme LLC, Rahway, NJ (US)

(72) Inventors: Blanca Homet Moreno, Phildelphia, PA (US); Nageatte Ibrahim, Plymouth Meeting, PA (US); Scot W. Ebbinghaus, Lansdale, PA (US); Lokesh Jain, Edison, NJ (US); Scott Diede, New Hope, PA (US)

(73) Assignee: Merck Sharp & Dohme LLC, Rahway, NJ (US)

(21) Appl. No.: 18/064,663

(22) Filed: Dec. 12, 2022

Related U.S. Application Data
(63) Continuation of application No. 16/499,674, filed on Sep. 30, 2019, now abandoned.
(60) Provisional application No. 62/479,784, filed on Mar. 31, 2017.

(51) Int. Cl. C07K 16/28; A61P 35/00

(52) U.S. Cl. CPC C07K 16/2818; A61K 2039/507; A61P 35/00

(57) ABSTRACT

The present invention relates to methods for treating cancer comprising administering an antagonist of PD-1, e.g., anti-PD-1 antibody or antigen binding fragment thereof, and an anti-CTLA4 antibody or antigen binding fragment thereof, wherein the CTLA4 antibody or antigen binding fragment thereof is given at a fixed dose. Also provided are compositions and kits comprising a dosage of an anti-PD-1 antibody and a dosage of an anti-CTLA4 antibody, and uses thereof.

### FIELD OF THE INVENTION

The present invention relates to combination therapies useful for the treatment of cancer. In particular, the invention relates to a combination therapy which comprises an antagonist of a Programmed Death 1 protein (PD-1) and a fixed dose of an anti-CTLA4 antibody or antigen binding fragment thereof.

### CROSS-REFERENCE TO RELATED APPLICATIONS

This application is a Continuation application of U.S. application Ser. No. 16/499,674, filed on Sep. 30, 2019, which is a national stage entry under 35 U.S.C. § 371 of PCT/US2018/024703 filed on Mar. 28, 2018, which claims priority to U.S. application 62/479,784 filed on Mar. 31, 2017, the contents of which are hereby incorporated by reference in their entireties.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Dec. 8, 2022, is named 24437-US-CNT-SEQ.XML and is 153 KB bytes in size.

### BACKGROUND OF THE INVENTION

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g., ovarian, renal, colorectal, pancreatic, liver cancers and melanoma, it was shown that PD-L1 expression correlated with poor prognosis and reduced overall survival irrespective of subsequent treatment (Dong et al., Nat Med. 8(8):793-800 (2002); Yang et al. Invest Ophthalmol Vis Sci. 49: 2518-2525 (2008); Ghebeh et al. Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953; Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J. Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al., BMC Cancer. 2008 8:5714-15 (2008); Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). Immune therapies targeting the PD-1 axis include monoclonal antibodies directed to the PD-1 receptor (KEYTRUDA™ (pembrolizumab), Merck and Co., Inc., Kenilworth, NJ, USA and OPDIVO™ (nivolumab), Bristol-Myers Squibb Company, Princeton, NJ, USA) and also those that bind to the PD-L1 ligand (MPDL3280A; TECENTRIQ™ (atezolizumab), Genentech, San Francisco, CA, USA). Both therapeutic approaches have demonstrated anti-tumor effects in numerous cancer types.

It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. One such agent that has been tested in combination with antagonists of PD-1 is cytotoxic T lymphocyte associated antigen 4 (abbreviated CTLA4).

CTLA4 has very close relationship with the CD28 molecule in gene structure, chromosome location, sequence homology and gene expression. Both of them are receptors for the co-stimulative molecule B7, mainly expressed on the surface of activated T cells. After binding to B7, CTLA4 can inhibit the activation of mouse and human T cells, playing a negative regulating role in the activation of T cells.

CTLA4 mAbs or CTLA4 ligands can prevent CTLA4 from binding to its native ligands, thereby blocking the transduction of the T cell negative regulating signal by CTLA4 and enhancing the responsiveness of T cells to various antigens. In this aspect, results from in vivo and in vitro studies are substantially in concert. At present, there are some CTLA4 mAbs being tested in clinical trials for treating prostate cancer, bladder cancer, colorectal cancer, cancer of gastrointestinal tract, liver cancer, malignant melanoma, etc. (Grosso et al., CTLA-4 blockade in tumor models: an overview of preclinical and translational research. Cancer Immun. 13:5 (2013)).

As important factors affecting the function of T cells, CTLA4 and CTLA4 mAbs can produce specific therapeutic effect on diseases by interfering with the immune microenvironment in the body. They have high efficacy and remedy the deficiency of traditional medication, opening a novel pathway of gene therapy. CTLA4 and CTLA4 mAbs are being tested in experiments and various stages of clinical trials. For example, in autoimmune diseases, they effectively inhibited airway hyperresponsiveness in an animal model of asthma, prevented the development of rheumatic diseases, mediated immune tolerance to an allograft in the body, and the like. On the other hand, although biological gene therapy has not shown any adverse effect in short term clinical trials, attention should be paid to the potential effect after long term application. For example, excessive blockade of CTLA4-B7 signaling by CTLA4 mAbs may result in the development of autoimmune diseases. As antibodies can specifically bind to their antigens and induce the lysis of target cells or block the progress of pathology, development and utilization of drugs based on antibodies, especially humanized antibodies have important significance in the clinical treatment of malignant tumors and other immune diseases in humans.

### SUMMARY OF THE INVENTION

The invention relates to methods for treating cancer in an individual comprising administering a combination of an antagonist of PD-1 (e.g. an anti-PD-1 antibody or antigen binding fragment thereof) and an anti-CTLA4 antibody or antigen binding fragment thereof, wherein the anti-CTLA4 antibody or binding fragment thereof is a reduced amount relative to the amount of the same antibody or antigen binding fragment thereof when administered as a monotherapy. In preferred embodiments, the dose of the anti-CTLA4 antibody or antigen binding fragment thereof is fixed, i.e., the dose does not depend on the individual's weight. The methods and compositions of the invention provide increased efficacy (e.g. an increased overall response rate) relative to administration of either agent as monotherapy and may provide improved safety and tolerability relative to treatment regimes comprising the combination, wherein each agent of the combination is given at the same dose as a monotherapy based on that agent.

Thus, in one embodiment, the invention provides a method of treating cancer in a human patient comprising administering: (a) 200 mg or 240 mg of an anti-PD-1 antibody or antigen binding fragment thereof and an anti-CTLA4 antibody, or antigen binding fragment thereof; or (b) 2 mg/kg of an anti-PD-1 antibody, or antigen binding fragment thereof, and an anti-CTLA4 antibody, or antigen binding fragment thereof, wherein the amount of the anti-CTLA4 antibody, or antigen binding fragment thereof, is selected from 10 mg, 25 mg, 50 mg, and 75 mg, and wherein the anti-PD-1 antibody, or antigen binding fragment thereof, is administered once every three weeks and the anti-CTLA4 antibody, or antigen binding fragment thereof, is administered once every three weeks or once every six weeks.

In another embodiment, the invention provides a method for treating cancer in a human patient comprising administering: (a) 200 mg or 240 mg of an anti-PD-1 antibody or antigen binding fragment thereof and 75 or 100 mg of an anti-CTLA4 antibody or antigen binding fragment thereof; or (b) 2 mg/kg of an anti-PD-1 antibody or antigen binding fragment thereof and 75 or 100 mg of an anti-CTLA4 antibody or antigen binding fragment thereof, wherein the anti-PD-1 antibody or antigen binding fragment thereof is administered once every three weeks and the anti-CTLA4 antibody or antigen binding fragment thereof is administered once every twelve weeks.

Also provided herein is a composition comprising a dosage of an anti-PD-1 antibody and a dosage of an anti-CTLA4 antibody, wherein the dosage is selected from the group consisting of: (1) 200 or 240 mg of an anti-PD-1 antibody and 10 mg of an anti-CTLA4 antibody; (2) 200 or 240 mg of an anti-PD-1 antibody and 25 mg of an anti-CTLA4 antibody; (3) 200 or 240 mg of an anti-PD-1 antibody and 50 mg of an anti-CTLA4 antibody; (4) 200 or 240 mg of an anti-PD-1 antibody and 75 mg of an anti-CTLA4 antibody; (5) 200 or 240 mg of an anti-PD-1 antibody and 100 mg of an anti-CTLA4 antibody; (6) 2 mg/kg of an anti-PD-1 antibody and 10 mg of an anti-CTLA4 antibody; (7) 2 mg/kg of an anti-PD-1 antibody and 25 mg of an anti-CTLA4 antibody; (8) 2 mg/kg of an anti-PD-1 antibody and 50 mg of an anti-CTLA4 antibody; (9) 2 mg/kg of an anti-PD-1 antibody and 75 mg of an anti-CTLA4 antibody; and (10) 2 mg/kg of an anti-PD-1 antibody and 100 mg of an anti-CTLA4 antibody.

The invention further provides kits for treating a patient with cancer, comprising: (a) a dosage of an anti-PD-1 antibody or antigen binding fragment thereof and a dosage of an anti-CTLA4 antibody or antigen binding fragment thereof, and (b) instructions for using the anti-PD-1 antibody or antigen binding fragment thereof and the anti-CTLA4 antibody or antigen binding fragment thereof in the methods of the invention.

The invention also relates to the use of the compositions, combinations, and kits for the treatment of cancer.

In all of the above treatment methods, compositions and uses, the PD-1 antagonist inhibits the binding of PD-L1 to PD-1, and preferably also inhibits the binding of PD-L2 to PD-1. In some preferred embodiments of the above treatment methods, compositions and uses, the PD-1 antagonist is a monoclonal antibody, or an antigen binding fragment thereof, which specifically binds to PD-1 or to PD-L1 and blocks the binding of PD-L1 to PD-1. In one particularly preferred embodiment, the PD-1 antagonist is an anti-PD-1 antibody which comprises a heavy chain and a light chain, and wherein the heavy and light chains comprise the amino acid sequences shown in FIG. 6 (SEQ ID NO:21 and SEQ ID NO:22) or wherein the heavy and light chains comprise the amino acid sequences shown in FIG. 7 (SEQ ID NO:23 and SEQ ID NO:24).

Also, in some embodiments of any of the above treatment methods, compositions and uses, the cancer expresses one or both of PD-L1 and PD-L2. In some embodiments, PD-L1 expression is elevated in the cancer.

### BRIEF DESCRIPTION OF THE DRAWINGS

FIG. 1 shows amino acid sequences of the light chain and heavy chain CDRs for an exemplary anti-PD-1 monoclonal antibody useful in the present invention (SEQ ID NOs:1-6).

FIG. 2 shows amino acid sequences of the light chain and heavy chain CDRs for another exemplary anti-PD-1 monoclonal antibody useful in the methods of the invention (SEQ ID NOs:7-12).

FIG. 3 shows amino acid sequences of the heavy chain variable region and full length heavy chain for an exemplary anti-PD-1 monoclonal antibody useful in the methods of the invention (SEQ ID NO:13 and SEQ ID NO:14).

FIG. 4 shows amino acid sequences of alternative light chain variable regions for an exemplary anti-PD-1 monoclonal antibody useful in the methods of the invention (SEQ ID NOs:15-17).

FIG. 5A shows amino acid sequences of alternative light chains for an exemplary anti-PD-1 monoclonal antibody useful in the methods of the invention (SEQ ID NOs: 18-19).

FIG. 5B shows an amino acid sequence of an alternative light chains for an exemplary anti-PD-1 monoclonal antibody useful in the methods of the invention (SEQ ID NO: 20).

FIG. 6 shows amino acid sequences of the heavy and light chains for pembrolizumab (SEQ ID NOs. 21 and 22, respectively). The V_{H} and V_{L} chain amino acid sequences are underlined and the CDR's are boxed (heavy chain CDRs are set forth in SEQ ID NOs:10, 11, and 12 and light chain CDRs are set forth in SEQ ID NOs:7, 8, and 9).

FIG. 7 shows amino acid sequences of the heavy and light chains for nivolumab (SEQ ID NOs. 23 and 24, respectively). The V_{H} and V_{L} chain amino acid sequences are underlined and the CDR's are boxed (heavy chain CDRs are set forth in SEQ ID NOs:28, 29, and 30 and light chain CDRs are set forth in SEQ ID NOs:31, 32, and 33).

FIG. 8 provides the study design for a method of treating cancer in accordance with the invention, as described in Example 2.

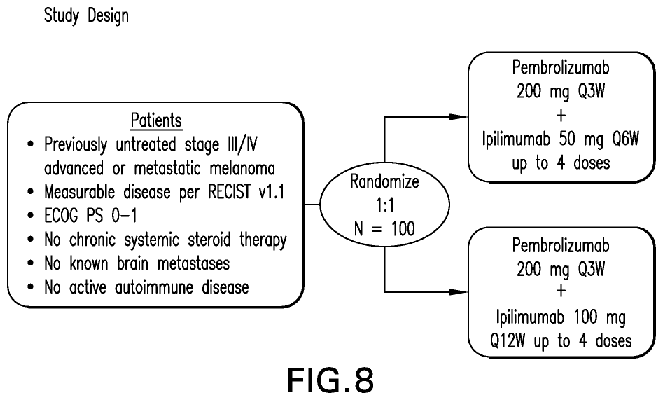

FIG. 9 provides the patient eligibility criteria for the study shown in FIG. 8 and described in Example 2.

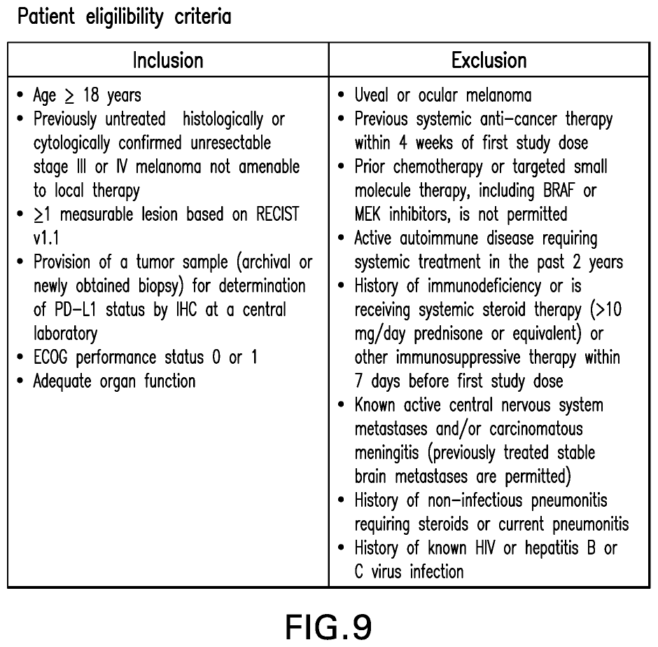

FIG. 10 shows amino acid sequences of the heavy and light chains for ipilimumab (SEQ ID NOs. 83 and 84, respectively).

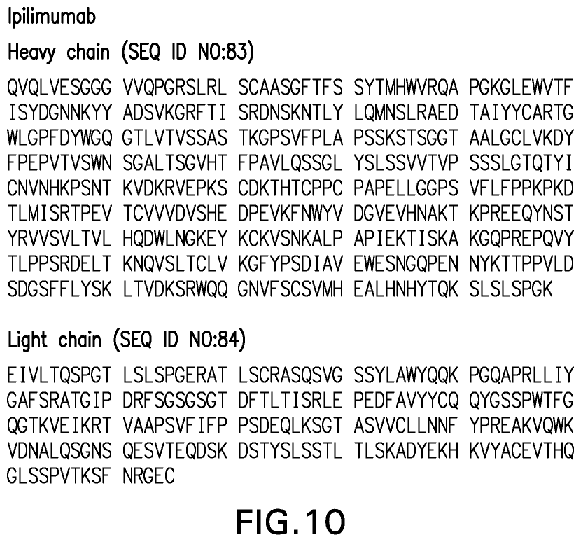

### DETAILED DESCRIPTION OF THE INVENTION

### I. Definitions and Abbreviations

As used throughout the specification and appended claims, the following abbreviations apply:
AE adverse event
CDR complementarity determining region
CHO Chinese hamster ovary
CR complete response
CTLA4 cytotoxic T lymphocyte associated antigen 4
DOR duration of response
FFPE formalin-fixed, paraffin-embedded
FR framework region
IgG immunoglobulin G
IHC immunohistochemistry or immunohistochemical
IPI ipilimumab
MEL melanoma
ORR overall response rate
OS overall survival
PD-1 programmed death 1 (a.k.a. programmed cell death-1 and programmed death receptor 1)
PD-L1 programmed cell death 1 ligand 1
PD-L2 programmed cell death 1 ligand 2
PD progressive disease
PFS progression free survival
PR partial response
Q3W one dose every three weeks
Q6W one dose every six weeks
Q12W one dose every twelve weeks
SD stable disease
TPS tumor proportion score
VGPR very good partial response
V_{H} immunoglobulin heavy chain variable region
V_{L} immunoglobulin light chain variable region

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Reference to “or” indicates either or both possibilities unless the context clearly dictates one of the indicated possibilities. In some cases, “and/or” was employed to highlight either or both possibilities.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Treat” or “treating” a cancer as used herein means to administer a combination therapy of a PD-1 antagonist and an anti-CTLA4 antibody or antigen binding fragment thereof to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating, abrogating the clinical manifestations of PD-1-related disease, reducing the severity or duration of the clinical symptoms of PD-1-related disease such as cancer, prolonging the survival of a patient relative to the expected survival in a similar untreated patient, and inducing complete or partial remission of a cancerous condition or other PD-1 related disease.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the present invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient.

The term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The term “hypervariable region” refers to the amino acid residues of an antibody that are responsible for antigen-binding. The hypervariable region comprises amino acid residues from a “complementarity determining region” or “CDR” (i.e., CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues refers to those variable domain residues other than the hypervariable region residues defined herein as CDR residues.

Unless otherwise indicated, an “antibody fragment” or “antigen binding fragment” refers to antigen binding fragments of antibodies, i.e., antibody fragments that retain the ability to specifically bind to the antigen bound by the full-length antibody, e.g., fragments that retain one or more CDR regions. Examples of antibody binding fragments include, but are not limited to, Fab, Fab′, F(ab′)₂, and Fv fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-1 or human PD-L1 molecule or the amino acid sequence of a mature human CTLA-4 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

An “anti-CTLA-4 antibody” means an antibody, or antigen binding fragment thereof, which binds to human CTLA-4 so as to disrupt the interaction of CTLA-4 with a human B7 receptor. After binding to B7, CTLA4 can inhibit the activation of mouse and human T cells, playing a negative regulating role in the activation of T cells. As used herein, unless specifically stated, said B7 refers to B7-1 and/or B7-2; and their specific protein sequences refer to the sequences known in the art. Reference can be made to the sequences disclosed in the literature or GenBank, e.g., B7-1 (CD80, NCBI Gene ID: 941), B7-2 (CD86, NCBI Gene ID: 942).

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Particularly preferred cancers that may be treated in accordance with the present invention include those characterized by elevated expression of one or both of PD-L1 and PD-L2 in tested tissue samples.

“Biotherapeutic agent” means a biological molecule, such as an antibody or fusion protein, that blocks ligand/receptor signaling in any biological pathway that supports tumor maintenance and/or growth or suppresses the anti-tumor immune response.

“CDR” or “CDRs” means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Chothia” means an antibody numbering system described in Al-Lazikani et al., JMB 273:927-948 (1997).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see. e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“Comprising” or variations such as “comprise”, “*comprises” ˜ or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Diagnostic anti-PD-L monoclonal antibody” means a mAb which specifically binds to the mature form of the designated PD-L (PD-L1 or PDL2) that is expressed on the surface of certain mammalian cells. A mature PD-L lacks the presecretory leader sequence, also referred to as leader peptide The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 25)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET .

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO 2014/100079. These antibodies comprise the light chain and heavy chain variable region amino acid sequences shown in Table 2 below:

TABLE 2
Monoclonal Antibodies 20C3 and 22C3
20C3 Light Chain Mature Variable Region

| | |
|---|---|
| DIVMSQSPSSLAVSAGEKVTMSCKSSQSLLNSRTRKNYLAWYQQ | SEQ ID NO: 103 |
| KPGQSPKLLIYWASTRESGVPDRFTGSGSGTDFTLTISSVQAED | |
| LAGTKLELKVYYCQQSYDVVTFGA | |

20C3 Heavy Chain Mature Variable Region

| | |
|---|---|
| QVQVQQSGAELAEPGASVKMSCKASGYIFTSYWMHWLKQRPGQ | SEQ ID NO: 104 |
| GLEWIGYINPSSDYNEYSEKFMDKATLTADKASTTAYMQLISLTS | |
| EDSAVYYCARSGWLVHGDYYFDYWGQGTTLTVSS | |

22C3 Light Chain Mature Variable Region

| | |
|---|---|
| DIVMSQSPSSLAVSAGEKVTMTCKSSQSLLHTSTRKNYLAWYQQ | SEQ ID NO: 105 |
| KPGQSPKLLIYWASTRESGVPDRFTGSGSGTDFTLTISSVQAEDLA | |
| VYYCKQSYDVVTFGAGTKLELK | |

22C3 Heavy Chain Mature Variable Region

| | |
|---|---|
| QVHLQQSGAELAKPGASVKMSCKASGYTFTSYWIHWIKQRPGQ | SEQ ID NO: 106 |
| GLEWIGYINPSSGYHEYNQKFIDKATLTADRSSSTAYMHLTSLTSE | |
| DSAVYYCARSGWLIHGDYYFDFWGQGTTLTVSS | |

Another anti-human PD-L1 mAb that has been reported to be useful for IHC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P.R. China; Catalog number 10084-R015).

“Framework region” or “FR” as used herein means the immunoglobulin variable regions excluding the CDR regions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see. e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in any of the treatment methods, compositions and uses of the present invention include an “anti-PD-1 antibody” and an “anti-PD-L1 antibody,” which both include monoclonal antibodies (mAb), or antigen binding fragments thereof, which specifically bind to human PD-1 and human PD-L1, respectively. An anti-PD-1 antibody and an anti-PD-L1 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and realtime quantitative RT-PCR.

Several approaches have been described for quantifying PD-L1 protein expression in IHC assays of tumor tissue sections. See, e.g., Thompson et al., PNAS 101 (49): 17174-17179 (2004); Thompson et al., Cancer Res. 66:3381-3385 (2006); Gadiot et al., Cancer 117:2192-2201 (2011); Taube et al., Sci Transl Med 4, 127ra37 (2012); and Toplian et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≥5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

A tissue section from a tumor that has been stained by IHC with a diagnostic PD-L1 antibody may also be scored for PD-L1 protein expression by assessing PD-L1 expression in both the tumor cells and infiltrating immune cells in the tissue section using a scoring process. See WO2014/165422. One PD-L1 scoring process comprises examining each tumor nest in the tissue section for staining, and assigning to the tissue section one or both of a modified H score (MHS) and a modified proportion score (MPS). To assign the MHS, four separate percentages are estimated across all of the viable tumor cells and stained mononuclear inflammatory cells in all of the examined tumor nests: (a) cells that have no staining (intensity=0), (b) weak staining (intensity=1+), (c) moderate staining (intensity=2+) and (d) strong staining (intensity=3+). A cell must have at least partial membrane staining to be included in the weak, moderate or strong staining percentages. The estimated percentages, the sum of which is 100%, are then input into the formula of 1×(percent of weak staining cells)+2×(percent of moderate staining cells)+3×(percent of strong staining cells), and the result is assigned to the tissue section as the MHS. The MPS is assigned by estimating, across all of the viable tumor cells and stained mononuclear inflammatory cells in all of the examined tumor nests, the percentage of cells that have at least partial membrane staining of any intensity, and the resulting percentage is assigned to the tissue section as the MPS. In some embodiments, the tumor is designated as positive for PD-L1 expression if the MHS or the MPS is positive.

The level of PD-L mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

### II. PD-1 Antagonists. Antibodies. And Antigen Binding Fragments and CTLA4 Antibodies and Antigen Binding Fragments Useful in the Invention

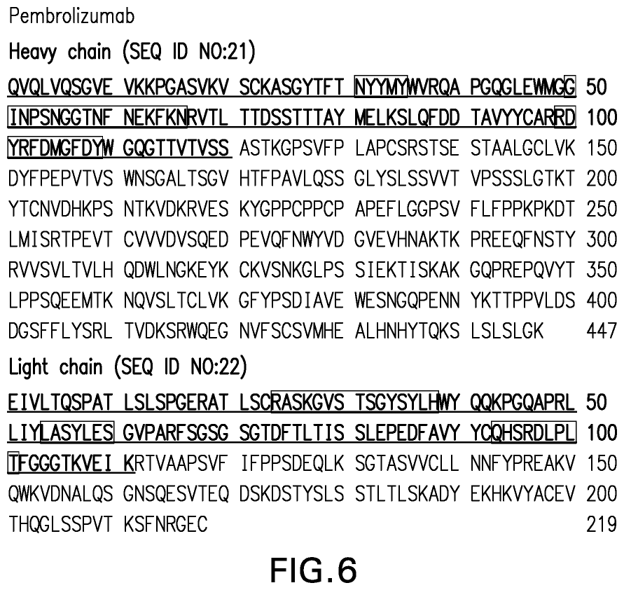

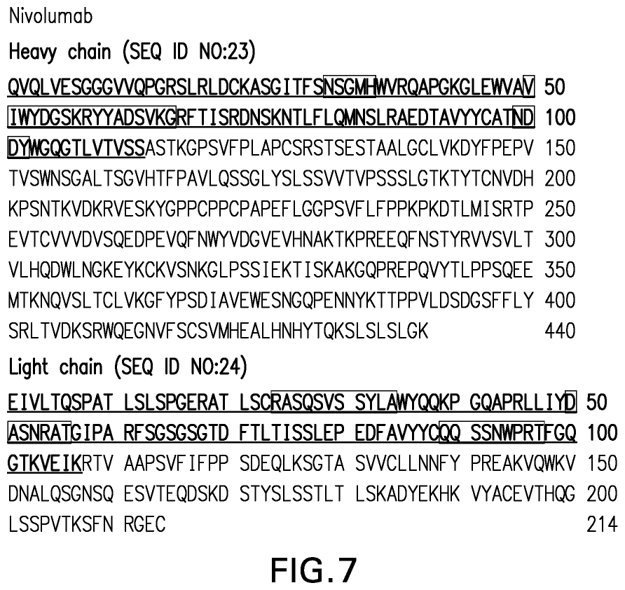

Examples of mAbs that bind to human PD-1, and useful in the treatment methods, compositions, kits and uses of the invention, are described in U.S. Pat. Nos. 7,521,051, 8,008,449, and 8,354,509. Specific anti-human PD-1 mAbs useful as the PD-1 antagonist in the treatment methods, compositions, kits and uses of the present invention include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 6, nivolumab (BMS-936558), a human IgG4 mAb with the structure described in WHO Drug Information, Vol. 27, No. 1, pages 68-69 (2013) and which comprises the heavy and light chain amino acid sequences shown in FIG. 7; pidilizumab (CT-011, also known as hBAT or hBAT-1); and the humanized antibodies h409A11, h409A16 and h409A17, which are described in WO2008/156712.

Examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include TECENTRIQ™ (atezolizumab, Genentech, San Francisco, CA, USA, formerly MPDL3280A), BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment methods, compositions, kits and uses of the invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some embodiments of the treatment methods, compositions and uses of the present invention, the PD-1 antagonist is a monoclonal antibody, or antigen binding fragment thereof, which comprises: (a) light chain CDRs comprising a sequence of amino acids as set forth in SEQ ID NOs: 1, 2 and 3 and heavy chain CDRs comprising a sequence of amino acids as set forth in SEQ ID NOs: 4, 5 and 6; or (b) light chain CDRs comprising a sequence of amino acids as set forth in SEQ ID NOs: 7, 8 and 9 and heavy chain CDRs comprising a sequence of amino acids as set forth in SEQ ID NOs: 10, 11 and 12.

In further embodiments of the treatment methods, compositions and uses of the present invention, the PD-1 antagonist is a monoclonal antibody, or antigen binding fragment thereof, which comprises: (a) light chain CDRs comprising a sequence of amino acids as set forth in SEQ ID NOs: 31, 32 and 33 and heavy chain CDRs comprising a sequence of amino acids as set forth in SEQ ID NOs: 28, 29 and 30.

In other embodiments of the treatment methods, compositions, kits and uses of the present invention, the PD-1 antagonist is a monoclonal antibody, or antigen binding fragment thereof, which specifically binds to human PD-1 and comprises (a) a heavy chain variable region comprising an amino acid sequence as set forth in SEQ ID NO:13 or a variant thereof, and (b) a light chain variable region comprising an amino acid sequence selected from the group consisting of SEQ ID NO:15 or a variant thereof; SEQ ID NO:16 or a variant thereof; and SEQ ID NO:17 or a variant thereof. In additional embodiments of the treatment methods, compositions, kits and uses of the present invention, the PD-1 antagonist is a monoclonal antibody, or antigen binding fragment thereof, which specifically binds to human PD-1 and comprises (a) a heavy chain variable region comprising an amino acid sequence as set forth in SEQ ID NO:26 or a variant thereof, and (b) a light chain variable region comprising an amino acid sequence as set forth in SEQ ID NO:27 or a variant thereof.

A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods, compositions, kits and uses of the present invention, the PD-1 antagonist is a monoclonal antibody which specifically binds to human PD-1 and comprises (a) a heavy chain comprising or consisting of a sequence of amino acids as set forth in SEQ ID NO: 14 and (b) a light chain comprising or consisting of a sequence of amino acids as set forth in SEQ ID NO:18, SEQ ID NO:19 or SEQ ID NO:20.

In yet another embodiment of the treatment methods, compositions, kits and uses of the invention, the PD-1 antagonist is a monoclonal antibody which specifically binds to human PD-1 and comprises (a) a heavy chain comprising or consisting of a sequence of amino acids as set forth in SEQ ID NO: 14 and (b) a light chain comprising or consisting of a sequence of amino acids as set forth in SEQ ID NO:18.

In further embodiments of the treatment methods, compositions, kits and uses of the invention, the PD-1 antagonist is a monoclonal antibody which specifically binds to human PD-1 and comprises (a) a heavy chain comprising or consisting of a sequence of amino acids as set forth in SEQ ID NO: 23 and (b) a light chain comprising or consisting of a sequence of amino acids as set forth in SEQ ID NO:24.

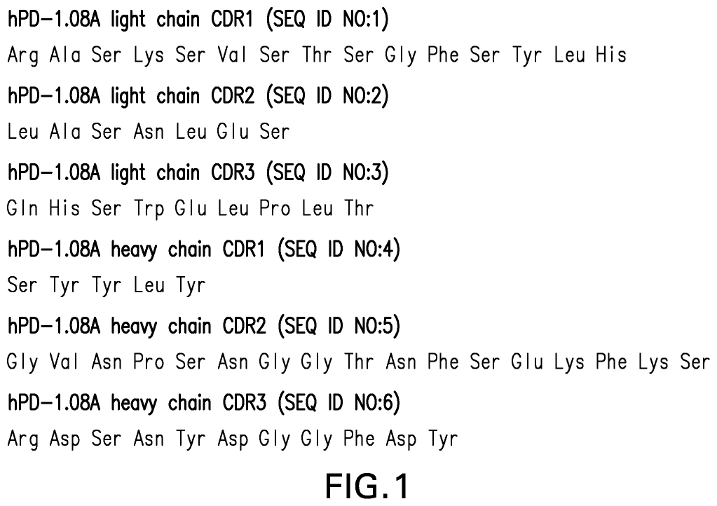

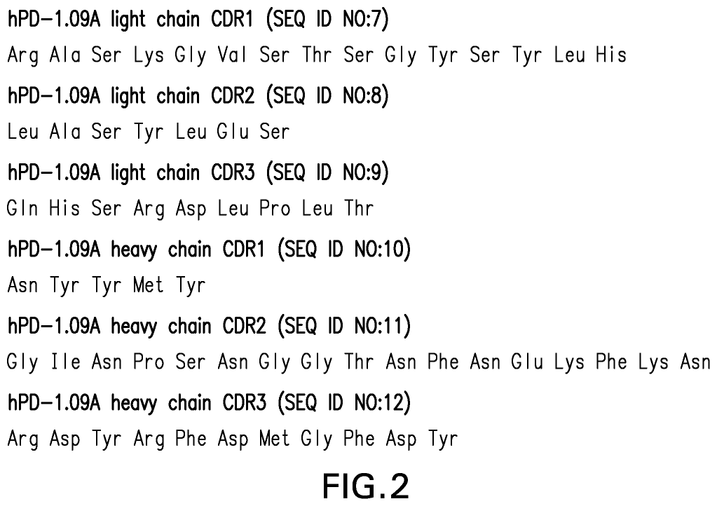

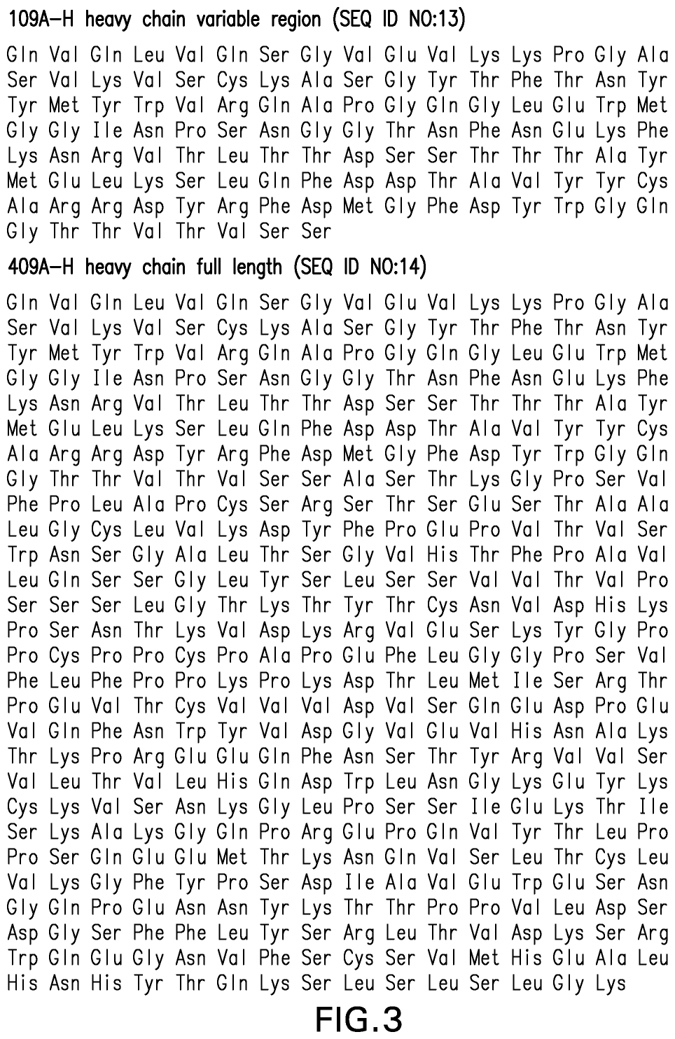

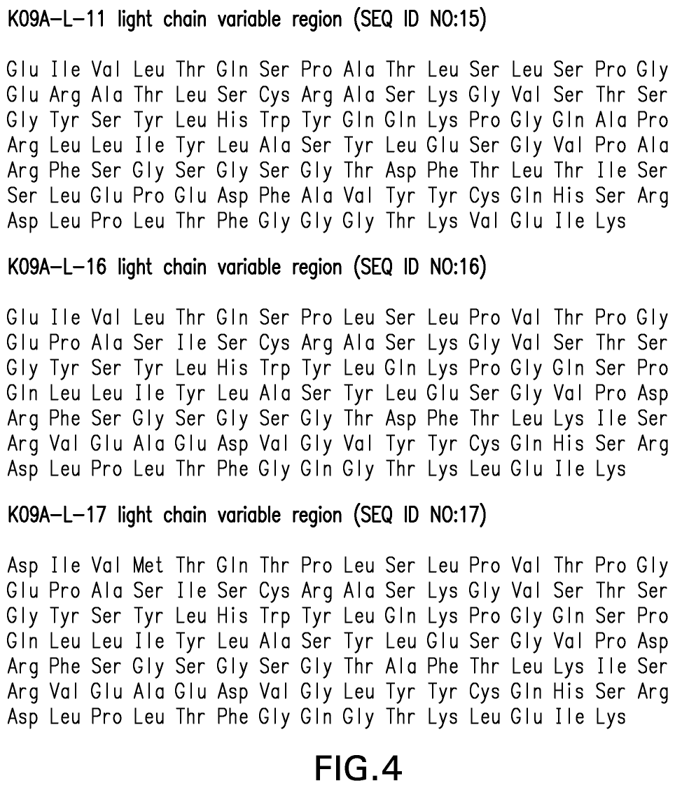

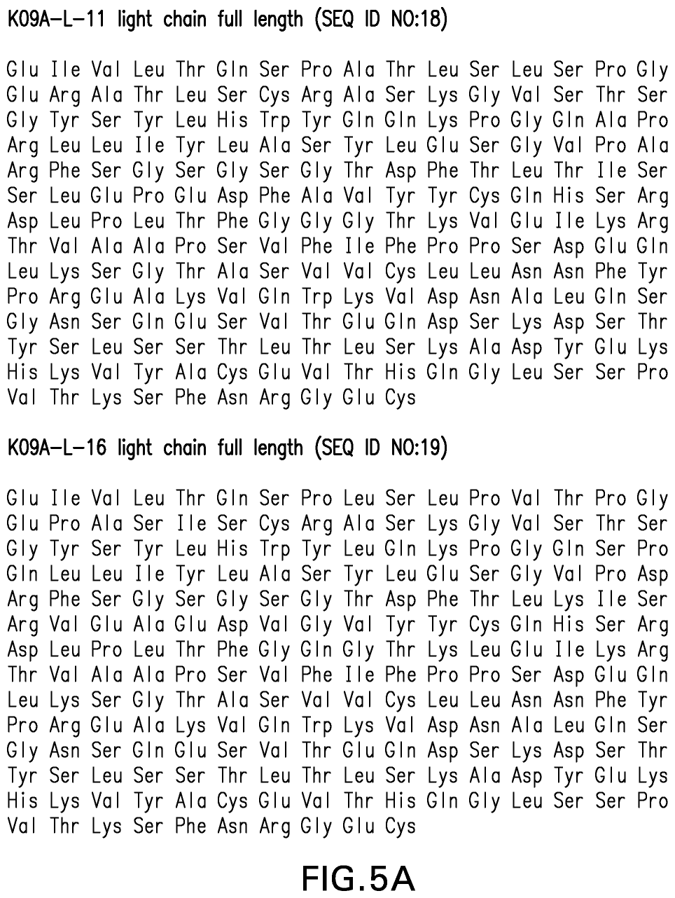

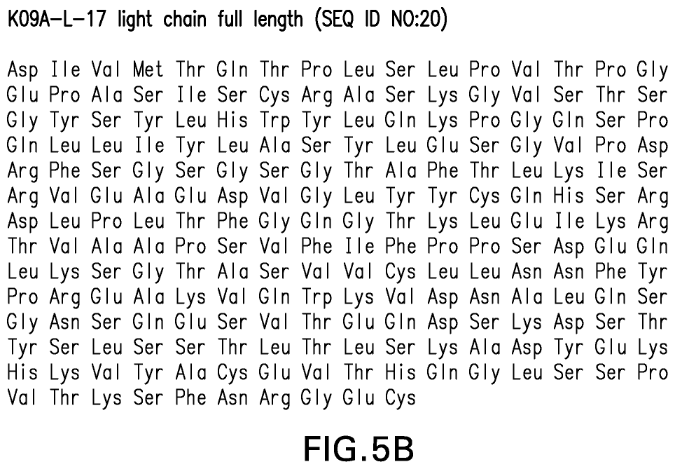

Table 3 below provides a list of the amino acid sequences of exemplary anti-PD-1 mAbs for use in the treatment methods, compositions, kits and uses of the present invention. The sequences are provided in FIGS. 1-5.

TABLE 3
Exemplary anti-human PD-1 antibodies
A. Comprises light and heavy chain CDRs of hPD-1.08A in WO2008/156712

| | |
|---|---|
| CDRL1 | SEQ ID NO: 1 |
| CDRL2 | SEQ ID NO: 2 |
| CDRL3 | SEQ ID NO: 3 |
| CDRH1 | SEQ ID NO: 4 |
| CDRH2 | SEQ ID NO: 5 |
| CDRH3 | SEQ ID NO: 6 |

B. Comprises light and heavy chain CDRs of hPD-1.09A in WO2008/156712

| | |
|---|---|
| CDRL1 | SEQ ID NO: 7 |
| CDRL2 | SEQ ID NO: 8 |
| CDRL3 | SEQ ID NO: 9 |
| CDRH1 | SEQ ID NO: 10 |
| CDRH2 | SEQ ID NO: 11 |
| CDRH3 | SEQ ID NO: 12 |

C. Comprises the mature h109A heavy chain variable region and one of
the mature K09A light chain variable regions in WO 2008/156712

| | |
|---|---|
| Heavy chain VR | SEQ ID NO: 13 |
| Light chain VR | SEQ ID NO: 15 or SEQ ID NO: 16 or SEQ ID NO: 17 |

D. Comprises the mature 409 heavy chain and one of the mature K09A
light chains in WO 2008/156712

| | |
|---|---|
| Heavy chain | SEQ ID NO: 14 |
| Light chain | SEQ ID NO: 18 or SEQ ID NO: 19 or SEQ ID NO: 20 |

In one embodiment of the treatment methods, compositions, kits and uses of the invention, the anti-CTLA-4 antibody is the human monoclonal antibody 10D1, now known as ipilimumab, and marketed as Yervoy™, which is disclosed in U.S. Pat. No. 6,984,720 and WHO Drug Information 19(4): 61 (2005). In another embodiment, the anti-CTLA-4 antibody is tremelimumnab, also known as CP-675,206, which is an IgG2 monoclonal antibody which is described in U.S. Patent Application Publication No. 2012/263677, or PCT International Application Publication Nos. WO 2012/122444 or 2007/113648 A2.

In further embodiments of the treatment methods, compositions and uses of the present invention, anti-CTLA4 antibody, or antigen binding fragment thereof, comprises: (a) light chain CDRs comprising a sequence of amino acids as set forth in SEQ ID NOs: 88, 89 and 90 and heavy chain CDRs comprising a sequence of amino acids as set forth in SEQ ID NOs: 85, 86 and 87.

In other embodiments of the treatment methods, compositions and uses of the present invention, the anti-CTLA4 antibody is a monoclonal antibody, or antigen binding fragment thereof, bind to human CTLA4 and comprises (a) a heavy chain variable region comprising an amino acid sequence as set forth in SEQ ID NO: 91 and (b) a light chain variable region comprising an amino acid sequence as set forth in SEQ ID NO: 92.

TABLE 4
Exemplary anti-human CTLA4 antibodies
A. Comprises light and heavy chain CDRs of Ipilimumab

| | |
|---|---|
| CDRL1 | RASQSVGSSYLA (SEQ ID NO: 88) |
| CDRL2 | GAFSRAT (SEQ ID NO: 89) |
| CDRL3 | QQYGSSPWT (SEQ ID NO: 90) |
| CDRH1 | SYTMH (SEQ ID NO: 85) |
| CDRH2 | FISYDGNNKYYADSVKG (SEQ ID NO: 86) |
| CDRH3 | TGWLGPFDY (SEQ ID NO: 87) |

C. Comprises the mature heavy chain variable region
and the mature light chain variable region of Ipilimumab

| | |
|---|---|
| Heavy chain VR | QVQLVESGGGVVQPGRSLRLSCAASGFTFSSYTMHWVRQA |
| | PGKGLEWVTFISYDGNNKYYADSVKGRFTISRDNSKNTLY |
| | LQMNSLRAEDTAIYYCARTGWLGPFDYWGQGTLVTVSS |
| | (SEQ ID NO: 91) |
| Light chain VR | EIVLTQSPGT LSLSPGERATLSCRASQSVGSSYLAWYQQK |
| | PGQAPRLLIYGAFSRATGIPDRFSGSGSGTDFTLTISRLE |
| | PEDFAVYYCQQYGSSPWTFGQGTKVEIK (SEQ ID NO: 92) |

D. Comprises the mature heavy chain and the mature light
chain of Ipilimumab

| | |
|---|---|
| Heavy chain | SEQ ID NO: 83 |
| Light chain | SEQ ID NO: 84 |

In one embodiment of the treatment methods, compositions, kits and uses of the invention, the anti-CTLA-4 antibody is a monoclonal antibody that comprises a heavy chain having the amino acid sequence set forth in SEQ ID NO: 83 and a light chain comprising the amino acid sequence set forth in SEQ ID NO: 84. In some embodiments, the CTLA4 antibody is an antigen binding fragment of SEQ ID NO: 83 and/or SEQ ID NO: 84, wherein the antigen binding fragment specifically binds to CTLA4.

In one embodiment of the treatment methods, compositions, kits and uses of the invention, the anti-CTLA-4 antibody is any of the anti-CTLA-4 antibodies, or antigen binding fragments thereof, disclosed in International Application Publication No. WO 2016/015675 A1. In one embodiment, the anti-CTLA4 antibody is a monoclonal antibody which comprises the following CDR's:
CDRH1 comprising the amino acid sequence GFTFSDNW (SEQ ID NO:34)
CDRH2 comprising the amino acid sequence IRNKPYNYET (SEQ ID NO:35)
CDRH3 comprising the amino acid sequence TAQFAY (SEQ ID NO:36) and/or
CDRL1 comprising the amino acid sequence ENIYGG (SEQ ID NO: 37)
CDRL2 comprising the amino acid sequence GAT (SEQ ID NO:38)
CDRL3 comprising an amino acid sequence selected from: QNVLRSPFT (SEQ ID NO:39); QNVLSRHPG (SEQ ID NO:40); OR QNVLSSRPG (SEQ ID NO:41)

In one embodiment of the treatment methods, compositions, kits and uses of the invention, the anti-CTLA4 antibody is 8D2/8D2 (RE) or a variant thereof, 8D2H1L1 or a variant thereof, 8D2H2L2 or a variant thereof, 8D3H3L3 or a variant thereof, 8D2H2L15 or a variant thereof, or 8D2H2117 or a variant thereof.

| Antibody | V_{H} | V_{L} |
|---|---|---|
| 8D2/8D2 | EVKLDETGGGLVQPGRPMKLSCVAS | DIQMTQSPASLSASVGETVTITCGT |
| (RE) | GFTFSDNWMNWVRQSPEKGLEWLA | SENIYGGLNWYQRKQGKSPQLLIF |
| | QIRNKPYNYETYYSDSVKGRFTISRD | GATNLADGMSSRFSGSGSGRQYSL |
| | DSKSSVYLQMNNLRGEDMGIYYCTA | KISSLHPDDVATYYCQNVLRSPFTF |
| | QFAYWGQGTLVTVSA (SEQ ID | GSGTKLEI (SEQ ID NO: 43) |
| | NO: 42) | |
| 8D2/8D2 | EVKLDETGGGLVQPGRPIKLSCVASG | DIQMTQSPASLSASVGETVTITCGT |
| RE | FTFSDNWMNWVRQSPEKGLEWLAQI | SENIYGGLNWYQRKQGKSPQLLIF |
| VARIANT1 | RNKPYNYETYYSDSVKGRFTISRDDS | GATNLADGMSSRFSGSGSGRQYSL |
| | KSSVYLQMNNLRGEDMGIYYCTAQF | KISSLHPDDVATYYCQNVLRSPFTF |
| | AYWGQGTLVTVSA (SEQ ID NO: 94) | GSGTKLEI (SEQ ID NO: 43) |
| 8D2H1L1 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKQGKSPKLLIY |
| | QIRNKPYNYETYYSDSVKGRFTISRD | GATNLASGMSSRFSGSGSGTDYTL |
| | DSKNSVYLQMNSLKTEDTGVYYCTA | KISSLHPDDVATYYCQNVLRSPFTF |
| | QFAYWGQGTLVTVSS (SEQ ID | GSGTKLEIK (SEQ ID NO: 45) |
| | NO: 44) | |
| 8D2HIL1 | EVQLVESGGGLVQPGGSIRLSCAASG | DIQMTQSPSSLSASVGDRVTITCRT |
| VARIANT1 | FTFSDNWMNWVRQAPGKGLEWLAQ | SENIYGGLNWYQRKQGKSPKLLIY |
| | IRNKPYNYETYYSDSVKGRFTISRDD | GATNLASGMSSRFSGSGSGTDYTL |
| | SKNSVYLQMNSLKTEDTGVYYCTAQ | KISSLHPDDVATYYCQNVLRSPFTF |
| | FAYWGQGTLVTVSS (SEQ ID NO: 96) | GSGTKLEIK (SEQ ID NO: 45) |
| 8D2H2L2 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLRSPFTF |
| | QFAYWGQGTLVTVSS (SEQ ID | GSGTKLEIK (SEQ ID NO: 47) |
| | NO: 46) | |
| 8D2H2L2 | EVQLVESGGGLVQPGGSIRLSCAASG | DIQMTQSPSSLSASVGDRVTITCRT |
| VARIANT1 | FTFSDNWMNWVRQAPGKGLEWLAQ | SENIYGGLNWYQRKPGKSPKLLIY |
| | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLRSPFTF |
| | FAYWGQGTLVTVSS (SEQ ID NO: 98) | GSGTKLEIK (SEQ ID NO: 47) |
| 8D3H3L3 | EVQLVESGGGLVQPGGSLRLSCAAS | DIQMTQSPSSLSASVGDRVTITCRA |
| | GFTFSDNWMNWVRQAPGKGLEWV | SENIYGGLNWYQQKPGKAPKLLIY |
| | AQIRNKPYNYETEY AASVKGRFTISR | GATSLASGVPSRFSGSGSGTDYTL |
| | DDSKNSAYLQMNSLKTEDTAVYYCT | TISSLQPEDFATYYCQNVLRSPFTF |
| | AQFAYWGQGTLVTVSS (SEQ ID | GSGTKLEIK (SEQ ID NO: 49) |
| | NO: 48) | |
| 8D2H2L15 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLSRHPG |
| | QFAYWGQGTLVTVSS (SEQ ID | FGSGTKLEIK (SEQ ID NO: 51) |
| | NO: 50) | |
| 8D2H2L15 | EVQLVESGGGLVQPGGSIRLSCAASG | DIQMTQSPSSLSASVGDRVTITCRT |
| VARIANT1 | FTFSDNWMNWVRQAPGKGLEWLAQ | SENIYGGLNWYQRKPGKSPKLLIY |
| | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLSRHPG |
| | FAYWGQGTLVTVSS (SEQ ID NO: | FGSGTKLEIK (SEQ ID NO: 51) |
| | 100) | |
| 8D2H2L17 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLSSRPGF |
| | QFAYWGQGTLVTVSS (SEQ ID | GSGTKLEIK (SEQ ID NO: 53) |
| | NO: 52) | |
| 8D2H2L17 | EVQLVESGGGLVQPGGSIRLSCAASG | DIQMTQSPSSLSASVGDRVTITCRT |
| VARIANT1 | FTFSDNWMNWVRQAPGKGLEWLAQ | SENIYGGLNWYQRKPGKSPKLLIY |
| | IRNKPYNYETYYSASVKGRFTISRDD | GATNLASGVSSRFSGSGSGTDYTL |
| | SKNSVYLQMNSLKTEDTGVYYCTAQ | TISSLQPEDVATYYCQNVLSSRPGF |
| | FAYWGQGTLVTVSS (SEQ ID NO: | GSGTKLEIK (SEQ ID NO: 53) |
| | 102) | |

In another embodiment of the treatment methods, compositions, kits and uses of the invention, the anti-CTLA4 antibody is a variant of 8D2/8D2 (RE), a variant of 8D2H1L1, a variant of 8D2H2L2, a variant of 8D2H2L15, or a variant of 8D2H2117, wherein the methionine (Met) at position 18 in the VH chain amino acid sequence is independently substituted with an amino acid selected from: Leucine (Leu), Valine (Val), Isoleucine (Ile) or Alanine (Ala). In embodiments of the invention, the anti-CTLA4 antibody is 8D2/8D2 (RE) Variant 1, 8D2H1L1 Variant 1, 8D2H2L2 Variant 1, 8D2H2L15 Variant 1, or 8D2H2117 Variant 1 as set forth in the table above. In another embodiment, the variant of 8D2/8D2 (RE), 8D2H1L1, 8D2H2L2, 8D2H2L15, and 8D2H2117 has a VH chain which comprises the amino acid sequence set forth in any one of SEQ ID NOs: 93, 94, 95, 96, 97, 98, 99, 100, 101, and 102.

| Antibody | V_{H} (Consensus) |
|---|---|
| 8D2/8D2 | EVKLDETGGGLVQPGRPXKLSCVASGFTFSDNWMNWVRQSPEK |
| (RE) | GLEWLAQIRNKPYNYETYYSDSVKGRFTISRDDSKSSVYLQMN |
| | NLRGEDMGIYYCTAQFAYWGQGTLVTVSA |
| | Wherein X = L (leu), V (val), I (Ile), or |
| | A (ala) |
| | (SEQ ID NO: 93) |
| 8D2HIL1 | EVQLVESGGGLVQPGGSXRLSCAASGFTFSDNWMNWVRQAPGK |
| | GLEWLAQIRNKPYNYETYYSDSVKGRFTISRDDSKNSVYLQMN |
| | SLKTEDTGVYYCTAQFAYWGQGTLVTVSS |
| | Wherein X = L (leu), V (val), I (Ile), or |
| | A (ala) |
| | (SEQ ID NO: 95) |
| 8D2H2L2 | EVQLVESGGGLVQPGGSXRLSCAASGFTFSDNWMNWVRQAPGK |
| | GLEWLAQIRNKPYNYETYYSASVKGRFTISRDDSKNSVYLQMN |
| | SLKTEDTGVYYCTAQFAYWGQGTLVTVSS |
| | Wherein X = L (leu), V (val), I (Ile), or |
| | A (ala) |
| | (SEQ ID NO: 97) |
| 8D2H2L15 | EVQLVESGGGLVQPGGSXRLSCAASGFTFSDNWMNWVRQAPGK |
| | GLEWLAQIRNKPYNYETYYSASVKGRFTISRDDSKNSVYLQMN |
| | SLKTEDTGVYYCTAQFAYWGQGTLVTVSS |
| | Wherein X = L (leu), V (val), I (Ile), or |
| | A (ala) |
| | (SEQ ID NO: 99) |
| 8D2H2L17 | EVQLVESGGGLVQPGGSXRLSCAASGFTFSDNWMNWVRQAPGK |
| | GLEWLAQIRNKPYNYETYYSASVKGRFTISRDDSKNSVYLQMN |
| | SLKTEDTGVYYCTAQFAYWGQGTLVTVSS |
| | Wherein X = L (leu), V (val), I (Ile), or |
| | A (ala) |
| | (SEQ ID NO: 101) |

In one embodiment of the treatment methods, compositions, kits and uses of the invention, the anti-CTLA4 antibody is any of the anti-CTLA4 antibodies, or antigen binding fragments thereof, described in disclosed in International Application Publication No. WO 2018/035710 A1, published Mar. 1, 2018. In one embodiment, the anti-CTLA4 antibody is mouse antibody 4G10, comprising the following VH chain and VL chain amino sequences, and humanized versions of this antibody.

| Antibody | V_{H} | V_{L} |
|---|---|---|
| 4G10 | QVKLQESGPELVKPGASMK | QAVVTQESALTTSPGETVTLT |
| murine | ISCKASGYSFTGYTMNWVK | CRSSTGAVTTSNFANWVQEKP |
| | QSHGKNLEWIGLINPYNNI | DHLFTSLIGGTNNRAPGVPAR |
| | TNYNQKFMGKATFTVDKSS | FSGSLIGDKAALTITGAQTED |
| | STAYMELLRLTSEDSGVYF | EAIYFCALWYSNHWVFGGGTK |
| | CARLDYRSYWGQGTLVTVS | LTVLGQPKSSPSVTLFQGQFC |
| | A (SEQ ID NO: 54) | (SEQ ID NO: 55) |

In one embodiment of the treatment methods, compositions, kits and uses of the invention, the anti-CTLA4 antibody is a monoclonal antibody which comprises the following CDR's:
CDRH1 comprising the amino acid sequence selected from GYSFTGYT (SEQ ID NO:56) or GYTX₁N (SEQ ID NO:57), wherein X₁ is M,V,L,I,G,A,S,T.
CDRH2 comprising the amino acid sequence selected from INPYNX₁IX₂, (SEQ ID NO:58) wherein X₁ is N, D or E, and X₂ is T, D, E, G or A; or LINPYNX₁IX₂NYX₃QKFX₄G (SEQ ID NO:59), wherein X₁ is N, D; X₂ is T, D, E, G, or A; X3 is A or N; and X4 is Q or M.
CDRH3 comprising the amino acid sequence selected from LDYRSY (SEQ ID NO:60) or ARLDYRSY Q (SEQ ID NO:61)
   and/or
CDRL1 comprising the amino acid sequence selected from TGAVTTSNF (SEQ ID NO:62), or GSSTGAVTVSNFX₁N (SEQ ID NO:63), wherein X₁ is P or A; CDRL2 comprising the amino acid sequence selected from GTN, or GTNNX₁AX₂ (SEQ ID NO:64), wherein X₁ is K, R or any amino acid except M or C; and X2 is S or P; CDRL3 comprising an amino acid sequence selected from ALX₁YSNHX₂(SEQ ID NO:65), wherein X₁ is W or any amino acid except M or C and X₂ is W or any amino acid except M or C; or ALX₁YSNHX₂V (SEQ ID NO:66) wherein X₁ is W or any amino acid except M or C and X₂ is W or any amino acid except M or C.

In another embodiment, the humanized VH sequences of the 4G10 antibody comprises any of the following VH sequences:

| Antibody | V_{H} |
|---|---|
| 4G10H1 | QVQLVESGAELVKPGASMKISCKASGYSFTGYTMNWVKQA |
| humanized | PGQGLEWIGLINPYNNITNYNQKFMGKATFTVDKSISTAY |
| | MELSRLTSDDSGVYFCARLDYRSYWGQGTLVTVSA |
| | (SEQ ID NO: 67) |
| 4G10H3 | QVQLVESGAEVKKPGASVKVSCKASGYSFTGYTMNWVRQA |
| humanized | PGQGLEWIGLINPYNNITNYAQKFQGRVTFTVDTSISTAY |
| | MELSRLRSDDTGVYFCARLDYRSYWGQGTLVTVSA |
| | (SEQ ID NO: 68) |
| 4G10H4 | QVQLVESGAEVKKPGASVKVSCKASGYSFTGYTMNWVRQA |
| humanized | PGQGLEWIGLINPYNDITNYAQKFQGRVTFTVDTSISTAY |
| | MELSRLRSDDTGVYFCARLDYRSYWGQGTLVTVSA |
| | (SEQ ID NO: 69) |
| 4G10H5 | QVQLVESGAEVKKPGASVKVSCKASGYSFTGYTMNWVRQA |
| humanized | PGQGLEWIGLINPYNNIDNYAQKFQGRVTFTVDTSISTAY |
| | MELSRLRSDDTGVYFCARLDYRSYWGQGTLVTVSA |
| | (SEQ ID NO: 70) |
| 4G10H | QVQLVESGAEX₁KKPGASX₂KX₃SCKASGYSFTGYTX₄NW |
| consensus | VX₅QAPGQGLEWIGLINPYNX₆IX₇NYX₈QKFX₉GX₁₀X₁₁ |
| humanized | TFTVDX₁₂SISTAYMELSRLX₁₃SDDX₁₄GVYFCARLDYRS |
| | YWGQGTLVTVSA (SEQ ID NO: 71) |
| | X₁ = V or L |
| | X₂ = V or M |
| | X₃ = V or I |
| | X₄ = M, V, L, I, G, A, S, T |
| | X₅ = R or K |
| | X₆ = N or D or E |
| | X₇ = T or D or E or G or A |
| | X₈ = A or N |
| | X₉ = Q or M |
| | X₁₀ = R or K |
| | X₁₁ = V or A |
| | X₁₂ = T or K |
| | X₁₃ = R or T |
| | X₁₄ = T or S |

In other embodiments of the treatment methods, compositions, kits and uses of the invention, the humanized VL sequences of the 4G10 antibody comprises any of the following VL sequences:

| Antibody | V_{L} |
|---|---|
| 4G10L1 | QAVVTQEPSLTVSPGGTVTLTCGSSTGAVTTSNFANWV |
| humanized | QEKPGQAFRSLIGGTNNRASWVPARFSGSLLGGKAALT |
| | ISGAQPEDEAEYFCALWYSNHWVFGGGTKLTVL |
| | (SEQ ID NO: 72) |
| 4G10L3 | QAVVTQEPSLTVSPGGTVTLTCGSSTGAVTTSNFPNWV |
| humanized | QQKPGQAPRSLIGGTNNKASWTPARFSGSLLGGKAALT |
| | ISGAQPEDEAEYYCALWYSNHWVFGGGTKLTVL |
| | (SEQ ID NO: 73) |
| 4G10Lconsensus | QAVVTQEPSLTVSPGGTVTLTCGSSTGAVTTSNFX₁NW |
| humanized | VQX₂KPGQAX₃RSLIGGTNNX₄AX₅WX₆PARFSGSLLGG |
| | KAALTISGAQPEDEAEYX₇CALX₈YSNHX₉VFGGGTKL |
| | TVL (SEQ ID NO: 74) |
| | X₁ = P or A |
| | X₂ = Q or E |
| | X₃ = Por F |
| | X₄ = K or R or any other amino acid |
| | except for M or C |
| | X₅ = S or P |
| | X₆ = T or V |
| | X₇ = Y or F |
| | X₈ = W or any amino acid except M or C |
| | X₉ = W or any amino acid except M or C |

In some embodiments, the anti-CTLA4 antibody is 4G10H1L1, 4G10H3L3, 4G10H3L3, and 4G10H5L3, each described below.

| Antibody | V_{H} | V_{L} |
|---|---|---|
| 4G10H1L1 | QVQLVESGAELVKPGASMKISCKA | QAVVTQEPSLTVSPGGTVTLTCGS |
| | SGYSFTGYTMNWVKQAPGQGLE | STGAVTTSNFANWVQEKPGQAFR |
| | WIGLINPYNNITNYNQKFMGKATF | SLIGGTNNRASWVPARFSGSLLG |
| | TVDKSISTAYMELSRLTSDDSGVY | GKAALTISGAQPEDEAEYFCALW |
| | FCARLDYRSYWGQGTLVTVSA | YSNHWVFGGGTKLTVL (SEQ ID |
| | (SEQ ID NO: 75) | NO: 76) |
| 4G10H3L3 | QVQLVESGAEVKKPGASVKVSCK | QAVVTQEPSLTVSPGGTVTLTCGS |
| | ASGYSFTGYTMNWVRQAPGQGLE | STGAVTTSNFPNWVQQKPGQAPR |
| | WIGLINPYNNITNYAQKFQGRVTF | SLIGGTNNKASWTPARFSGSLLGG |
| | TVDTSISTAYMELSRLRSDDTGVY | KAALTISGAQPEDEAEYYCALWY |
| | FCARLDYRSYWGQGTLVTVSA | SNHWVFGGGTKLTVL (SEQ ID |
| | (SEQ ID NO: 77) | NO: 78) |
| 4G10H4L3 | QVQLVESGAEVKKPGASVKVSCK | QAVVTQEPSLTVSPGGTVTLTCGS |
| | ASGYSFTGYTMNWVRQAPGQGLE | STGAVTTSNFPNWVQQKPGQAPR |
| | WIGLINPYNDITNYAQKFQGRVTF | SLIGGTNNKASWTPARFSGSLLGG |
| | TVDTSISTAYMELSRLRSDDTGVY | KAALTISGAQPEDEAEYYCALWY |
| | FCARLDYRSYWGQGTLVTVSA | SNHWVFGGGTKLTVL |
| | (SEQ ID NO: 79) | (SEQ ID NO: 80) |
| 4G10H5L3 | QVQLVESGAEVKKPGASVKVSCK | QAVVTQEPSLTVSPGGTVTLTCGS |
| | ASGYSFTGYTMNWVRQAPGQGLE | STGAVTTSNFPNWVQQKPGQAPR |
| | WIGLINPYNNIDNYAQKFQGRVTF | SLIGGTNNKASWTPARFSGSLLGG |
| | TVDTSISTAYMELSRLRSDDTGVY | KAALTISGAQPEDEAEYYCALWY |
| | FCARLDYRSYWGQGTLVTVSA | SNHWVFGGGTKLTVL |
| | (SEQ ID NO: 81) | (SEQ ID NO: 82) |

In another embodiment of the treatment methods, compositions, kits and uses of the invention, the anti-CTLA-4 antibody is an antibody, or antigen binding fragment thereof, which cross-competes for binding to human CTLA-4 with, or binds to the same epitope region of human CTLA-4 as does ipilimumab, tremelimumab, or any of the above described antibodies, including 8D2/8D2 (RE) or variant thereof, 8D2H1L1 or variant thereof, 8D2H2L2 or variant thereof, 8D3H3L3 or variant thereof, 8D2H2L15 or variant thereof, 8D2H2L17 or variant thereof, 4G10H1L1 or variant thereof, 4G10H3L3 or variant thereof, 4G10H3L3 or variant thereof, and 4G10H5L3 or variant thereof.

### III. Methods and Uses of the Invention

The invention provides a method of treating cancer in a human patient comprising administering: (a) 200 mg or 240 mg of an anti-PD-1 antibody or antigen binding fragment thereof and an anti-CTLA4 antibody or antigen binding fragment thereof; or (b) 2 mg/kg of an anti-PD-1 antibody or antigen binding fragment thereof and an anti-CTLA4 antibody or antigen binding fragment thereof, wherein the amount of the anti-CTLA4 antibody or antigen binding fragment thereof is selected from 10 mg, 25 mg, 50 mg, and 75 mg, and wherein the anti-PD-1 antibody or antigen binding fragment thereof is administered once every three weeks and the anti-CTLA4 antibody or antigen binding fragment thereof is administered once every three weeks or once every six weeks (Method 1).

In one embodiment, the method comprises administering 200 mg of an anti-PD-1 antibody and 10 mg of an anti-CTLA4 antibody.

In another embodiment, the method comprises administering 240 mg of an anti-PD-1 antibody and 10 mg of an anti-CTLA4 antibody.

In a further embodiment, the method comprises administering 2 mg/kg of an anti-PD-1 and 10 mg of an anti-CTLA4 antibody.

In one embodiment, the method comprises administering 200 mg of an anti-PD-1 antibody and 25 mg of an anti-CTLA4 antibody.

In another embodiment, the method comprises administering 240 mg of an anti-PD-1 antibody and 25 mg of an anti-CTLA4 antibody.

In a further embodiment, the method comprises administering 2 mg/kg of an anti-PD-1 and 25 mg of an anti-CTLA4 antibody.

In one embodiment, the method comprises administering 200 mg of an anti-PD-1 antibody and 50 mg of an anti-CTLA4 antibody.

In another embodiment, the method comprises administering 240 mg of an anti-PD-1 antibody and 50 mg of an anti-CTLA4 antibody.

In a further embodiment, the method comprises administering 2 mg/kg of an anti-PD-1 and 50 mg of an anti-CTLA4 antibody.

In one embodiment, the method comprises administering 200 mg of an anti-PD-1 antibody and 75 mg of an anti-CTLA4 antibody.

In another embodiment, the method comprises administering 240 mg of an anti-PD-1 antibody and 75 mg of an anti-CTLA4 antibody.

In a further embodiment, the method comprises administering 2 mg/kg of an anti-PD-1 and 75 mg of an anti-CTLA4 antibody.

Also provided are methods for treating cancer as described above with alternative dosing. In said methods, the dose of the anti-PD-1 antibody or antigen binding fragment thereof is from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-PD-1 antibody or antigen binding fragment thereof is 150 mg, 175 mg, 200 mg, 225 mg, 240 mg, or 250 mg.

In one embodiment of any of the above methods, the dose of the anti-CTLA4 antibody or antigen binding fragment thereof is 10 mg, 25 mg, 50 mg, or 75 mg.

In some embodiments of any of the methods above, the anti-CTLA4 antibody or antigen binding fragment thereof is administered once every six weeks for 24 weeks or less.

In alternative embodiments of any of the methods above, the anti-CTLA4 antibody or antigen binding fragment thereof is administered once every three weeks for 24 weeks or less.

Also provided herein is a method as described in any of the above embodiments, the method further comprising continuing the administration of the anti-PD-1 antibody once every three weeks after administration of the anti-CTLA4 antibody is discontinued.

Also provided by the invention is a method of treating cancer in a human patient comprising administering: (a) 200 mg or 240 mg of an anti-PD-1 antibody or antigen binding fragment thereof and 75 or 100 mg of an anti-CTLA4 antibody or antigen binding fragment thereof; or (b) 2 mg/kg of an anti-PD-1 antibody or antigen binding fragment thereof and 75 or 100 mg of an anti-CTLA4 antibody or antigen binding fragment thereof, wherein the anti-PD-1 antibody or antigen binding fragment thereof is administered once every three weeks and the anti-CTLA4 antibody or antigen binding fragment thereof is administered once every twelve weeks (Method 2).

One embodiment of Method 2 comprises administering 200 mg of an anti-PD-1 antibody and 100 mg of an anti-CTLA4 antibody.

Another embodiment, comprises administering 240 mg of an anti-PD-1 antibody and 100 mg of an anti-CTLA4 antibody.

One embodiment comprises administering 2 mg/kg of an anti-PD-1 antibody and 100 mg of an anti-CTLA4 antibody.

In sub-embodiments of Method 2, the anti-CTLA antibody or antigen binding fragment thereof is administered once every twelve weeks for 48 weeks or less.

Also provided herein is a method as described in any of the above embodiments, the method further comprising continuing the administration of the anti-PD-1 antibody once every three weeks after administration of the anti-CTLA4 antibody is discontinued.

In any of the methods of the invention described above (Method 1 or Method 2), the PD-1 antibody or antigen binding fragment and the CTLA4 antibody or antigen binding fragment can be any of the antibodies or antigen-binding fragments described in Section II of the Detailed Description of the Invention “PD-1 Antagonists, Antibodies, and Antigen Binding Fragments and CTLA4 Antibodies and Antigen Binding Fragments Useful in the Invention” herein.

In some embodiments, the anti-PD-1 antibody is pembrolizumab or an antigen-binding fragment thereof, nivolumab or an antigen binding fragment thereof, or an antibody which cross competes with pembrolizumab or nivolumab for binding to human PD-1.

In some embodiments, the anti-CTLA4 antibody is selected from the group consisting of:
a) ipilimumab,
b) tremelimumab,
c) 8D2/8D2 (RE), 8D2/8D2 (RE) Variant 1, or other variant thereof,
d) 8D2H1L1, 8D2H1L1 Variant 1, or other variant thereof,
e) 8D2H2L2, 8D2H2L2 Variant 1, or other variant thereof,
f) 8D3H3L3, or variant thereof,
g) 8D2H2L15, 8D2H2L15 Variant 1, or other variant thereof,
h) 8D2H2L17, 8D2H2L17 Variant 1, or other variant thereof,
i) 4G10H1L1,
j) 4G10H3L3,
k) 4G10H3L3,
l) 4G10H5L3, and
m) an antibody which cross competes with ipilimumab or tremelimumab for binding to human CTLA-4;
   wherein the methionine at position 18 in the VH chain amino acid sequence of the 8D2/8D2 (RE) variant, the 8D2H1L1 variant, the 8D2H2L2 variant, the 8D3H3L3 variant, the 8D2H2L15 variant, and the 8D2H2L17 variant is independently substituted with an amino acid selected from: leucine, valine, isoleucine, and alanine.

In specific embodiments, the anti-CTLA4 antibody is 8D2/8D2 (RE), 8D2/8D2 (RE) Variant 1, or other variant of 8D2/8D2 (RE); 8D2H1L1, 8D2H1L1 Variant 1, or other variant of 8D2H1L1; 8D2H2L2, 8D2H2L2 Variant 1, or other variant of 8D2H2L2; 8D3H3L3 or variant thereof; 8D2H2L15, 8D2H2L15 Variant 1, or other variant of 8D2H2L15; or 8D2H2L17, 8D2H2L17 Variant 1, or other variant of 8D2H2L17.

In some specific embodiments, the anti-CTLA4 antibody is a variant of 8D2/8D2 (RE), a variant of 8D2H1L1, a variant of 8D2H2L2, a variant of 8D2H2L15, or a variant of 8D2H2L17, wherein the amino acid at position 18 of the VH sequence of the anti-CTLA4 antibody is isoleucine. In other embodiments, the anti-CTLA4 antibody is a 8D2/8D2 (RE) Variant 1, 8D2H1L1 Variant 1, 8D2H2L2 Variant 1, 8D2H2L15 Variant 1, or 8D2H2L17 Variant 1.

In some embodiments of the methods of the invention, the anti-PD-1 antibody is pembrolizumab and the anti-CTLA4 antibody is ipilimumab.

In some embodiments of the methods of the invention, the anti-PD-1 antibody is pembrolizumab and the anti-CTLA4 antibody is tremelimumab.

In some embodiments of the methods of the invention, the anti-PD-1 antibody is pembrolizumab and the anti-CTLA4 antibody is 8D2/8D2 (RE), 8D2/8D2 (RE) Variant 1 or a other variant of 8D2/8D2 (RE).

In some embodiments of the methods of the invention, the anti-PD-1 antibody is pembrolizumab and the anti-CTLA4 antibody is 8D2H1L1, 8D2H1L1 Variant 1, or a other variant of 8D2H1L1.

In some embodiments of the methods of the invention, the anti-PD-1 antibody is pembrolizumab and the anti-CTLA4 antibody is 8D2H2L2, 8D2H2L2 Variant 1, or other variant of 8D2H2L2.

In some embodiments of the methods of the invention, the anti-PD-1 antibody is pembrolizumab and the anti-CTLA4 antibody is 8D3H3L3 or a variant thereof.

In some embodiments of the methods of the invention, the anti-PD-1 antibody is pembrolizumab and the anti-CTLA4 antibody is 8D2H2L15, 8D2H2L15 Variant 1, or other variant of 8D2H2L15.

In some embodiments of the methods of the invention, the anti-PD-1 antibody is pembrolizumab and the anti-CTLA4 antibody is 8D2H2L17, 8D2H2L17 Variant 1, or other variant of 8D2H2L17.

In some embodiments of the methods of the invention, the anti-PD-1 antibody is pembrolizumab and the anti-CTLA4 antibody is 4G10H1L1.

In some embodiments of the methods of the invention, the anti-PD-1 antibody is pembrolizumab and the anti-CTLA4 antibody is 4G10H3L3.

In some embodiments of the methods of the invention, the anti-PD-1 antibody is pembrolizumab and the anti-CTLA4 antibody is 4G10H4L3.

In some embodiments of the methods of the invention, the anti-PD-1 antibody is pembrolizumab and the anti-CTLA4 antibody is 4G10H5L3.

In some embodiments of the methods of the invention, the anti-PD-1 antibody is pembrolizumab and the anti-CTLA4 antibody is an antibody which cross competes with ipilimumab or tremelimumab for binding to human CTLA-4.

In some embodiments of the methods of the invention, the anti-PD-1 antibody is nivolumab and the anti-CTLA4 antibody is ipilimumab.

In some embodiments of the methods of the invention, the anti-PD-1 antibody is nivolumab and the anti-CTLA4 antibody is tremelimumab.

In some embodiments of the methods of the invention, the anti-PD-1 antibody is nivolumab and the anti-CTLA4 antibody is 8D2/8D2 (RE), 8D2/8D2 (RE) Variant 1, or a other variant of 8D2/8D2 (RE).

In some embodiments of the methods of the invention, the anti-PD-1 antibody is nivolumab and the anti-CTLA4 antibody is, 8D2H1L1 Variant 1, or a other variant of 8D2H1L1.

In some embodiments of the methods of the invention, the anti-PD-1 antibody is nivolumab and the anti-CTLA4 antibody is 8D2H2L2, 8D2H2L2 Variant 1, or a other variant of 8D2H2L2.

In some embodiments of the methods of the invention, the anti-PD-1 antibody is nivolumab and the anti-CTLA4 antibody is 8D3H3L3 or a variant thereof.

In some embodiments of the methods of the invention, the anti-PD-1 antibody is nivolumab and the anti-CTLA4 antibody is 8D2H2L15, 8D2H2L15 Variant 1, or a other variant of 8D2H2L15.

In some embodiments of the methods of the invention, the anti-PD-1 antibody is nivolumab and the anti-CTLA4 antibody is 8D2H2L17, 8D2H2L17 Variant 1, or a other variant of 8D2H2L17.

In some embodiments of the methods of the invention, the anti-PD-1 antibody is nivolumab and the anti-CTLA4 antibody is 4G10H1L1.

In some embodiments of the methods of the invention, the anti-PD-1 antibody is nivolumab and the anti-CTLA4 antibody is 4G10H3L3.

In some embodiments of the methods of the invention, the anti-PD-1 antibody is nivolumab and the anti-CTLA4 antibody is 4G10H4L3.

In some embodiments of the methods of the invention, the anti-PD-1 antibody is nivolumab and the anti-CTLA4 antibody is 4G10H5L3.

In some embodiments of the methods of the invention, the anti-PD-1 antibody is nivolumab and the anti-CTLA4 antibody is an antibody which cross competes with ipilimumab or tremelimumab for binding to human CTLA-4.

In any of the methods of the invention, the cancer is selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma.

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer.

In some embodiments, the cancer is metastatic colorectal cancer with high levels of microsatellite instability (MSI-H).

In some embodiments, the cancer is selected from the group consisting of: melanoma, non-small cell lung cancer, relapsed or refractory classical Hodgkin lymphoma, head and neck squamous cell carcinoma, urothelial cancer, esophageal cancer, gastric cancer, and hepatocellular cancer.

In other embodiments of the above treatment methods, the cancer is a Heme malignancy. In certain embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin lymphoma (NHL), or small lymphocytic lymphoma (SLL).

In embodiments of any of the methods herein, a subject is administered an intravenous (IV) infusion of a medicament comprising any of the PD-1 antagonists described herein and a medicament comprising any of the anti-CTLA4 antibodies or antigen binding fragments thereof.

The combination therapy may also comprise one or more “additional therapeutic agents” (as used herein, “additional therapeutic agent” refers to an additional agent relative to the PD-1 antagonist and the anti-CTLA4 antibody or antigen binding fragment thereof). The additional therapeutic agent may be, e.g., a chemotherapeutic other than an anti-PD-1 antibody or an anti-CTLA4 antibody, a biotherapeutic agent (including but not limited to antibodies to VEGF, EGFR, Her2/neu, VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In some embodiments which comprise a step of administering an additional therapeutic agent (i.e., in addition to the PD-1 antagonist and the anti-CTLA4 antibody or binding fragment thereof), the additional therapeutic agent in the combination therapy may be administered using the same dosage regimen (dose, frequency and duration of treatment) that is typically employed when the agent is used as monotherapy for treating the same cancer. In other embodiments, the patient receives a lower total amount of the additional therapeutic agent in the combination therapy than when that agent is used as monotherapy, e.g., smaller doses, less frequent doses, and/or shorter treatment duration.

The additional therapeutic agent in a combination therapy can be administered orally or parenterally, including the intravenous, intramuscular, intraperitoneal, subcutaneous, rectal, topical, and transdermal routes of administration. For example, the combination treatment may comprise an anti-PD-1 antibody or antigen binding fragment thereof, and an anti-CTLA antibody or antigen binding fragment thereof, both of which may be administered intravenously, as well as a chemotherapeutic agent, which may be administered orally.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention may be used to treat a tumor that is large enough to be found by palpation or by imaging techniques well known in the art, such as MRI, ultrasound, or CAT scan. In some embodiments, a combination therapy of the invention is used to treat an advanced stage tumor having dimensions of at least about 200 mm³, 300 mm³, 400 mm³, 500 mm³, 750 mm³, or up to 1000 mm³.

In some embodiments, a combination therapy of the invention is administered to a human patient who has a cancer that tests positive for PD-L1 expression. In some embodiments, PD-L1 expression is detected using a diagnostic anti-human PD-L1 antibody, or antigen binding fragment thereof, in an IHC assay on an FFPE or frozen tissue section of a tumor sample removed from the patient. A patient's physician may order a diagnostic test to determine PD-L1 expression in a tumor tissue sample removed from the patient prior to initiation of treatment with the PD-1 antagonist and the anti-CTLA4 antibody, but it is envisioned that the physician could order the first or subsequent diagnostic tests at any time after initiation of treatment, such as for example after completion of a treatment cycle.

Selecting a dosage of the additional therapeutic agent may include depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g. biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See. e.g., Wawrzynczak (1996) Antibody Therapy, Bios Scientific Pub. Ltd, Oxfordshire, UK; Kresina (ed.) (1991) Monoclonal Antibodies. Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

### IV. Compositions and Kits

The invention also relates to compositions comprising a dosage of a PD-1 antagonist, e.g., an anti-PD-1 antibody or antigen binding fragment thereof and a dosage of an anti-CTLA4 antibody or antigen binding fragment thereof, wherein the dosage is selected from the group consisting of:
i) 200 mg or 240 mg of an anti-PD-1 antibody or antigen binding fragment thereof and 10 mg of an anti-CTLA4 antibody or antigen binding fragment thereof;
ii) 200 mg or 240 mg of an anti-PD-1 antibody or antigen binding fragment thereof and 25 mg of an anti-CTLA4 antibody or antigen binding fragment thereof;
iii) 200 mg or 240 mg of an anti-PD-1 antibody or antigen binding fragment thereof and 50 mg of an anti-CTLA4 antibody or antigen binding fragment thereof;
iv) 200 mg or 240 mg of an anti-PD-1 antibody or antigen binding fragment thereof and 75 mg of an anti-CTLA4 antibody or antigen binding fragment thereof;
v) 200 mg or 240 mg of an anti-PD-1 antibody or antigen binding fragment thereof and 100 mg of an anti-CTLA4 antibody or antigen binding fragment thereof;
vi) 2 mg/kg of an anti-PD-1 antibody or antigen binding fragment thereof and 10 mg of an anti-CTLA4 antibody or antigen binding fragment thereof;
vii) 2 mg/kg of an anti-PD-1 antibody or antigen binding fragment thereof and 25 mg of an anti-CTLA4 antibody or antigen binding fragment thereof;
viii) 2 mg/kg of an anti-PD-1 antibody or antigen binding fragment thereof and 50 mg of an anti-CTLA4 antibody or antigen binding fragment thereof;
ix) 2 mg/kg of an anti-PD-1 antibody or antigen binding fragment thereof and 75 mg of an anti-CTLA4 antibody or antigen binding fragment thereof; and
x) 2 mg/kg of an anti-PD-1 antibody or antigen binding fragment thereof and 100 mg of an anti-CTLA4 antibody or antigen binding fragment thereof.

The present invention also provides a composition which comprises a PD-1 antagonist as described above and a pharmaceutically acceptable excipient. When the PD-1 antagonist is a biotherapeutic agent, e.g., a mAb, the antagonist may be produced in CHO cells using conventional cell culture and recovery/purification technologies.

In one embodiment, the invention provides a composition comprising a dosage of an anti-PD-1 antibody and a dosage of an anti-CTLA4 antibody, wherein the dosage is selected from the group consisting of:
i) 200 mg of an anti-PD-1 antibody and 10 mg of an anti-CTLA4 antibody;
ii) 200 mg of an anti-PD-1 antibody and 25 mg of an anti-CTLA4 antibody;
iii) 200 mg of an anti-PD-1 antibody and 50 mg of an anti-CTLA4 antibody;
iv) 200 mg of an anti-PD-1 antibody and 75 mg of an anti-CTLA4 antibody; and
v) 200 mg of an anti-PD-1 antibody and 100 mg of an anti-CTLA4 antibody.

In the compositions of the invention, the PD-1 antibody or antigen binding fragment thereof and the anti-CTLA4 antibody or antigen binding fragment thereof can be any of the antibodies and antigen binding fragments described herein, i.e., described in Section II of the Detailed Description of the Invention “PD-1 Antagonists, Antibodies, and Antigen Binding Fragments Useful in the Invention.”

In some embodiments, a composition comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention. In some preferred embodiments, a medicament comprising pembrolizumab is provided in a glass vial which contains about 50 mg of pembrolizumab.

In some embodiments of the compositions herein, the anti-PD-1 antibody is pembrolizumab, nivolumab, or an antibody which cross competes with pembrolizumab or nivolumab for binding to human PD-1.

In some embodiments, the anti-CTLA4 antibody is ipilimumab.

In further embodiments of the compositions of the invention, the anti-CTLA4 antibody is selected from the group consisting of:
a) ipilimumab,
b) tremelimumab,
c) 8D2/8D2 (RE), 8D2/8D2 (RE) Variant 1, or other a variant thereof,
d) 8D2H1L1, or a other variant thereof,
e) 8D2H2L2, 8D2H2L2, or a other variant thereof,
f) 8D3H3L3 or a variant thereof,
g) 8D2H2L15, 8D2H2L15, or a other variant thereof,
h) 8D2H2L17, 8D2H2L17, or a other variant thereof,
i) 4G10H1L1,
j) 4G10H3L3,
k) 4G10H4L3,
l) 4G10H5L3, and
m) an antibody which cross competes with ipilimumab or tremelimumab for binding to human CTLA-4;
   wherein the methionine at position 18 in the variable heavy (VH) chain amino acid sequence of the 8D2/8D2 (RE) variant, the 8D2H1L1 variant, the 8D2H2L2 variant, the 8D3H3L3 variant, the 8D2H2L15 variant, and the 8D2H2L17 variant is independently substituted with an amino acid selected from: leucine, valine, isoleucine, and alanine.

In some embodiments of the compositions of the invention, the anti-PD-1 antibody is pembrolizumab and the anti-CTLA4 antibody is ipilimumab.

In some embodiments of the compositions of the invention, the anti-PD-1 antibody is pembrolizumab and the anti-CTLA4 antibody is tremelimumab.

In some embodiments of the compositions of the invention, the anti-PD-1 antibody is pembrolizumab and the anti-CTLA4 antibody is 8D2/8D2 (RE), 8D2/8D2 (RE) Variant 1, or other variant of 8D2/8D2 (RE).

In some embodiments of the compositions of the invention, the anti-PD-1 antibody is pembrolizumab and the anti-CTLA4 antibody is 8D2H1L1, 8D2H1L1 Variant 1 or a other variant of 8D2H1L1.

In some embodiments of the compositions of the invention, the anti-PD-1 antibody is pembrolizumab and the anti-CTLA4 antibody is 8D2H2L2, 8D2H2L2 Variant 1 or a other variant of 8D2H2L2.

In some embodiments of the compositions of the invention, the anti-PD-1 antibody is pembrolizumab and the anti-CTLA4 antibody is 8D3H3L3 or a variant thereof.

In some embodiments of the compositions of the invention, the anti-PD-1 antibody is pembrolizumab and the anti-CTLA4 antibody is 8D2H2L15, 8D2H2L15 Variant 1, or a other variant of 8D2H2L15.

In some embodiments of the compositions of the invention, the anti-PD-1 antibody is pembrolizumab and the anti-CTLA4 antibody is 8D2H2L17, 8D2H2L17 Variant 1, or a other variant of 8D2H2L17.

In some embodiments of the compositions of the invention, the anti-PD-1 antibody is pembrolizumab and the anti-CTLA4 antibody is 4G10H1L1.

In some embodiments of the compositions of the invention, the anti-PD-1 antibody is pembrolizumab and the anti-CTLA4 antibody is 4G10H3L3.

In some embodiments of the compositions of the invention, the anti-PD-1 antibody is pembrolizumab and the anti-CTLA4 antibody is 4G10H4L3.

In some embodiments of the compositions of the invention, the anti-PD-1 antibody is pembrolizumab and the anti-CTLA4 antibody is 4G10H5L3.

In some embodiments of the compositions of the invention, the anti-PD-1 antibody is pembrolizumab and the anti-CTLA4 antibody is an antibody which cross competes with ipilimumab or tremelimumab for binding to human CTLA-4.

In some embodiments of the compositions of the invention, the anti-PD-1 antibody is nivolumab and the anti-CTLA4 antibody is ipilimumab.

In some embodiments of the compositions of the invention, the anti-PD-1 antibody is nivolumab and the anti-CTLA4 antibody is tremelimumab.

In some embodiments of the compositions of the invention, the anti-PD-1 antibody is nivolumab and the anti-CTLA4 antibody is 8D2/8D2 (RE), 8D2/8D2 (RE) Variant 1, or a other variant of 8D2/8D2 (RE).

In some embodiments of the compositions of the invention, the anti-PD-1 antibody is nivolumab and the anti-CTLA4 antibody is 8D2H1L1, 8D2H1L1 Variant 1, or a other variant of 8D2H1L1.

In some embodiments of the compositions of the invention, the anti-PD-1 antibody is nivolumab and the anti-CTLA4 antibody is 8D2H2L2, 8D2H2L2 Variant 1, or a other variant of 8D2H2L2.

In some embodiments of the compositions of the invention, the anti-PD-1 antibody is nivolumab and the anti-CTLA4 antibody is 8D3H3L3 or a variant thereof.

In some embodiments of the compositions of the invention, the anti-PD-1 antibody is nivolumab and the anti-CTLA4 antibody is 8D2H2L15, 8D2H2L15 Variant 1, or a other variant of 8D2H2L15.

In some embodiments of the compositions of the invention, the anti-PD-1 antibody is nivolumab and the anti-CTLA4 antibody is 8D2H2L17, 8D2H2L17 Variant 1, or a other variant of 8D2H2L17.

In some embodiments of the compositions of the invention, the anti-PD-1 antibody is nivolumab and the anti-CTLA4 antibody is 4G10H1L1.

In some embodiments of the compositions of the invention, the anti-PD-1 antibody is nivolumab and the anti-CTLA4 antibody is 4G10H3L3.

In some embodiments of the compositions of the invention, the anti-PD-1 antibody is nivolumab and the anti-CTLA4 antibody is 4G10H4L3.

In some embodiments of the compositions of the invention, the anti-PD-1 antibody is nivolumab and the anti-CTLA4 antibody is 4G10H5L3.

In some embodiments of the compositions of the invention, the anti-PD-1 antibody is nivolumab and the anti-CTLA4 antibody is an antibody which cross competes with ipilimumab or tremelimumab for binding to human CTLA-4.

In some embodiments of the compositions of the invention, the anti-PD-1 antibody is nivolumab and the anti-CTLA-4 antibody is selected from 8D2/8D2 (RE), 8D2/8D2 (RE) Variant 1, or other variant of 8D2/8D2 (RE); 8D2H1L1, 8D2H1L1 Variant 1, or other variant of 8D2H1L1; 8D2H2L2, 8D2H2L2 Variant 1, or other variant of 8D2H2L2; 8D3H3L3 or variant thereof; 8D2H2L15, 8D2H2L15 Variant 1, or other variant of 8D2H2L15; or 8D2H2L17, 8D2H2L17 Variant 1, or other variant of 8D2H2L17.

In other embodiments of the compositions of the invention, the anti-PD-1 antibody is pembrolizumab and the anti-CTLA4 antibody is the 8D2/8D2 (RE) Variant 1, 8D2H1L1 Variant 1, 8D2H2L2 Variant 1, 8D2H2L15 Variant 1, or 8D2H2L17 Variant 1.

The invention also relates to a kit for treating a patient with cancer, the kit comprising: (a) a dosage of an anti-PD-1 antibody or antigen binding fragment thereof and a dosage of an anti-CTLA4 antibody or antigen binding fragment thereof selected from the group consisting of: (i) 200 mg of an anti-PD-1 antibody or antigen binding fragment thereof and 10 mg of an anti-CTLA4 antibody or antigen binding fragment thereof; (ii) 200 mg of an anti-PD-1 antibody or antigen binding fragment thereof and 25 mg of an anti-CTLA4 antibody or antigen binding fragment thereof; (iii) 200 mg of an anti-PD-1 antibody or antigen binding fragment thereof and 50 mg of an anti-CTLA4 antibody or antigen binding fragment thereof; (iv) 200 mg of an anti-PD-1 antibody or antigen binding fragment thereof and 75 mg of an anti-CTLA4 antibody or antigen binding fragment thereof; and (v) 200 mg of an anti-PD-1 antibody or antigen binding fragment thereof and 100 mg of an anti-CTLA4 antibody or antigen binding fragment thereof; and (b) instructions for using the anti-PD-1 antibody or antigen binding fragment thereof and the anti-CTLA4 antibody or antigen binding fragment thereof in the method of any of claims 1-16.

In any of the kits of the invention, the PD-1 antibody or antigen binding fragment and the CTLA4 antibody or antigen binding fragment can be any of the antibodies or antigen-binding fragments described in Section II of the Detailed Description of the Invention “PD-1 Antagonists, Antibodies, and Antigen Binding Fragments Useful in the Invention”.

In some embodiments, the anti-PD-1 antibody is pembrolizumab, nivolumab, or an antibody which cross competes with pembrolizumab or nivolumab for binding to human PD-1.

In some embodiments, the anti-CTLA4 antibody is selected from the group consisting of: ipilimumab, tremelimumab, 8D2/8D2 (RE), 8D2/8D2 (RE) Variant 1, or a other variant of 8D2/8D2 (RE); 8D2H1L1, 8D2H1L1 Variant 1, or a other variant of 8D2H1L1; 8D2H2L2, 8D2H2L2, or a other variant of 8D2H2L2, 8D3H3L3 or a variant thereof; 8D2H2L15, 8D2H2L15 Variant 1, or a other variant of 8D2H2L15; 8D2H2L17, 8D2H2L17 Variant 1, or a other variant of 8D2H2L17, 4G10H1L1, 4G10H3L3, 4G10H3L3, 4G10H5L3, and an antibody which cross competes with ipilimumab or tremelimumab for binding to human CTLA-4, wherein the methionine at position 18 in the variable heavy (VH) chain amino acid sequence of the 8D2/8D2 (RE) variant, the 8D2H1L1 variant, the 8D2H2L2 variant, the 8D3H3L3 variant, the 8D2H2L15 variant, and the 8D2H2L17 variant is independently substituted with an amino acid selected from: leucine, valine, isoleucine, and alanine.

In some embodiments of the kits of the invention, the anti-PD-1 antibody is pembrolizumab and the anti-CTLA4 antibody is ipilimumab.

In some embodiments of the kits of the invention, the anti-PD-1 antibody is pembrolizumab and the anti-CTLA4 antibody is tremelimumab.

In some embodiments of the kits of the invention, the anti-PD-1 antibody is pembrolizumab and the anti-CTLA4 antibody is 8D2/8D2 (RE), 8D2/8D2 (RE) Variant 1, or a other variant of 8D2/8D2 (RE).

In some embodiments of the kits of the invention, the anti-PD-1 antibody is pembrolizumab and the anti-CTLA4 antibody is 8D2H1L1, 8D2H1L1 Variant 1, or a other variant of 8D2H1L1.

In some embodiments of the kits of the invention, the anti-PD-1 antibody is pembrolizumab and the anti-CTLA4 antibody is 8D2H2L2, 8D2H2L2 Variant 1, or a other variant of 8D2H2L2.

In some embodiments of the kits of the invention, the anti-PD-1 antibody is pembrolizumab and the anti-CTLA4 antibody is 8D3H3L3 or a variant thereof.

In some embodiments of the kits of the invention, the anti-PD-1 antibody is pembrolizumab and the anti-CTLA4 antibody is 8D2H2L15, 8D2H2L15 Variant 1, or a other variant of 8D2H2L15.

In some embodiments of the kits of the invention, the anti-PD-1 antibody is pembrolizumab and the anti-CTLA4 antibody is 8D2H2L17, 8D2H2L17 Variant 1, or a other variant of 8D2H2L17.

In some embodiments of the kits of the invention, the anti-PD-1 antibody is pembrolizumab and the anti-CTLA4 antibody is 4G10H1L1.

In some embodiments of the kits of the invention, the anti-PD-1 antibody is pembrolizumab and the anti-CTLA4 antibody is 4G10H3L3.

In some embodiments of the kits of the invention, the anti-PD-1 antibody is pembrolizumab and the anti-CTLA4 antibody is 4G10H4L3.

In some embodiments of the kits of the invention, the anti-PD-1 antibody is pembrolizumab and the anti-CTLA4 antibody is 4G10H5L3.

In some embodiments of the kits of the invention, the anti-PD-1 antibody is pembrolizumab and the anti-CTLA4 antibody is an antibody which cross competes with ipilimumab or tremelimumab for binding to human CTLA-4.

In some embodiments of the kits of the invention, the anti-PD-1 antibody is nivolumab and the anti-CTLA4 antibody is ipilimumab.

In some embodiments of the kits of the invention, the anti-PD-1 antibody is nivolumab and the anti-CTLA4 antibody is tremelimumab.

In some embodiments of the kits of the invention, the anti-PD-1 antibody is nivolumab and the anti-CTLA4 antibody is 8D2/8D2 (RE), 8D2/8D2 (RE) Variant 1, or a other variant of 8D2/8D2 (RE).

In some embodiments of the kits of the invention, the anti-PD-1 antibody is nivolumab and the anti-CTLA4 antibody is 8D2H1L1, 8D2H1L1 Variant 1, or a other variant of 8D2H1L1.

In some embodiments of the kits of the invention, the anti-PD-1 antibody is nivolumab and the anti-CTLA4 antibody is 8D2H2L2, 8D2H2L2 Variant 1 or a other variant of 8D2H2L2.

In some embodiments of the kits of the invention, the anti-PD-1 antibody is nivolumab and the anti-CTLA4 antibody is 8D3H3L3 or a variant thereof.

In some embodiments of the kits of the invention, the anti-PD-1 antibody is nivolumab and the anti-CTLA4 antibody is 8D2H2L15, 8D2H2L15 Variant 1 or a other variant of 8D2H2L15.

In some embodiments of the kits of the invention, the anti-PD-1 antibody is nivolumab and the anti-CTLA4 antibody is 8D2H2L17, 8D2H2L17 Variant 1, or a other variant of 8D2H2L17.

In some embodiments of the kits of the invention, the anti-PD-1 antibody is nivolumab and the anti-CTLA4 antibody is 4G10H1L1.

In some embodiments of the kits of the invention, the anti-PD-1 antibody is nivolumab and the anti-CTLA4 antibody is 4G10H3L3.

In some embodiments of the kits of the invention, the anti-PD-1 antibody is nivolumab and the anti-CTLA4 antibody is 4G10H4L3.

In some embodiments of the kits of the invention, the anti-PD-1 antibody is nivolumab and the anti-CTLA4 antibody is 4G10H5L3.

In some embodiments of the kits of the invention, the anti-PD-1 antibody is nivolumab and the anti-CTLA4 antibody is an antibody which cross competes with ipilimumab or tremelimumab for binding to human CTLA-4.

In some embodiments of the compositions of the invention, the anti-PD-1 antibody is nivolumab and the anti-CTLA-4 antibody is selected from 8D2/8D2 (RE), 8D2/8D2 (RE) Variant 1, or other variant of 8D2/8D2 (RE); 8D2H1L1, 8D2H1L1 Variant 1, or other variant of 8D2H1L1; 8D2H2L2, 8D2H2L2 Variant 1, or other variant of 8D2H2L2; 8D3H3L3 or variant thereof; 8D2H2L15, 8D2H2L15 Variant 1, or other variant of 8D2H2L15; or 8D2H2L17, 8D2H2L17 Variant 1, or other variant of 8D2H2L17.

In other embodiments of the compositions of the invention, the anti-PD-1 antibody is pembrolizumab and the anti-CTLA4 antibody is the 8D2/8D2 (RE) Variant 1, 8D2H1L1 Variant 1, 8D2H2L2 Variant 1, 8D2H2L15 Variant 1, or 8D2H2L17 Variant 1.

The kits of the invention may provide the anti-PD-1 and anti-CTLA4 antibodies or antigen-binding fragments thereof in a first container and a second container and a package insert. The first container contains at least one dose of a medicament comprising an anti-PD-1 antagonist, the second container contains at least one dose of a medicament comprising an anti-CTLA4 antibody or antigen binding fragment thereof, and the package insert, or label, which comprises instructions for treating a patient with cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some preferred embodiments of the kit, the anti-PD-1 antagonist is an anti-PD-1 antibody and the instructions state that the medicaments are intended for use in treating a patient having a tumor, wherein the tumor expresses PD-L1 by, e.g., an IHC assay. In some embodiments, the tumor has a tumor proportion score (TPS) of ≥1% PD-L1. In another embodiment, the tumor has a TPS of ≥50% PD-L1. A PD-L1 TPS is the number of tumor cells in a sample expressing PD-L1. In further embodiments, the tumor has a TPS of ≥5% PD-L1, ≥10 PD-L1, ≥15% PD-L1, ≥20% PD-L1, ≥25% PD-L1, ≥30% PD-L1, ≥35% PD-L1, ≥40% PD-L1, or ≥45% PD-L1.

These and other aspects of the invention, including the exemplary specific embodiments listed below, will be apparent from the teachings contained herein.

### General Methods

Standard methods in molecular biology are described Sambrook, Fritsch and Maniatis (1982 & 1989 2^{nd} Edition, 2001 3^{rd} Edition) Molecular Cloning. A Laboratory Manual, Cold Spring Harbor Laboratory Press, Cold Spring Harbor, NY; Sambrook and Russell (2001) Molecular Cloning. 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science. Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science. Vol. 2, John Wiley and Sons, Inc., New York; Ausubel, et al. (2001) Current Protocols in Molecular Biology. Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology. Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000)Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry. 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY). Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLE 1

A Phase I/II Study to characterize the safety, tolerability, and preliminary efficacy of the combination pembrolizumab+Ipilimumab in melanoma subjects.

Pre-clinical studies have shown that concomitant CTLA-4 and PD-1 blockage elicited greater response rates than either single agent in MC38 mouse models (Selby et al. Antitumor activity of concurrent blockade of immune checkpoint molecules CTLA-4 and PD-1 in preclinical models. [Abstract 3061]. 2013 ASCO Annual Meeting, General Poster Session, Developmental Therapeutics—Immunotherapy; 2013 May 31-Jun. 3. Chicago, IL, 2013). Notably, while no mice treated with anti-CTLA-4 or anti-PD-1 were free of disease at the end of the experiment, 7-60% of mice treated with the combination at various dose levels were tumor free.

In a Phase 1 trial in subjects with MEL, IPI (anti-CTLA-4) combined with nivolumab (anti-PD-1) led to response rates ranging from 21-53%. These may represent greater responses than nivolumab monotherapy (Sznol et al. [Abstract 3734]. European Society for Medical Oncology 2013 Annual Meeting; Sep. 27-Oct. 1, 2013; Amsterdam, Netherlands; Topalian et al. [Abstract 3002]. 2013 ASCO Annual Meeting; 2013 May 31-Jun. 4. Chicago, IL) and historical responses to IPI in monotherapy (approximately 20-31% and 10%, respectively) (Hodi et al., N Engl J Med 363(8):711-23 (2010)). Both anti-PD-1 antibodies nivolumab and pembrolizumab demonstrated superiority over single agent IPI in previously untreated subjects (pembrolizumab), and over chemotherapy both in untreated subjects and after failure of IPI (nivolumab, pembrolizumab). The addition of IPI to nivolumab is associated with a higher response rate and a better PFS, however, it is associated with a high rate of Grade 3-4 DRAEs.

In this study, pembrolizumab was evaluated in combination with IPI 1 mg/kg. The schedule of IPI followed the approved drug label of q3w. In a previous Phase I study efficacy of nivolumab 3 mg/kg+IPI 1 mg/kg (ORR 40% (95% CI 16-68%) and Aggregate Clinical Activity Rate (ACAR; defined as CR+PR+unconfirmed CR+unconfirmed PR+immune-related PR+SD >24 week+irSD >24 week) 73%] appeared to have similar efficacy as nivolumab 1 mg/kg+IPI 3 mg/kg (ORR 53% (95% CI 28-77%) and ACAR 65%) (Sznol et al.. Combined nivolumab (anti-PD-1, BMS-936558, ONO-4538) and ipilimumab in the treatment of advanced melanoma patients: Safety and clinical activity. [Abstract 3734]. European Society for Medical Onocology 2013 Annual Meeting; Sep. 27-Oct. 1, 2013; Amsterdam, Netherlands). In addition, the combination of nivolumab 3 mg/kg+IPI 1 mg/kg showed lower rate of Grade 3-4 DRAEs compared with nivolumab 1 mg/kg+IPI 3 mg/kg (44% versus 65%, respectively). A total of 6/28 (21%) of patients experienced Grade 3-4 toxicities that were found to be dose-limiting.

The overall dosing strategy of this study was intended to emphasize dose intensity with pembrolizumab rather than IPI; in addition, the dosing paradigm of anti-PD-1+anti-CTLA4 combination explored the lowest therapeutic dose of anti-PD-1 (pembrolizumab at 2 mg/kg q3w) and a lower dose than recommended monotherapy dose of anti-CTLA4 (IPI at 1 mg/kg q3w×4 doses).

Results of 153 MEL patients indicate that lower doses of IPI (1 mg/kg q3w) in combination with pembrolizumab 2 mg/kg q3w had a confirmed ORR of 57% by independent central review and a comparable rate of Grade 3-4 DRAEs to nivolumab 3 mg/kg+IPI 1 mg/kg (42 vs 44%) (Larkin et al., Combined Nivolumab and Ipilimumab or Monotherapy in Untreated Melanoma. N Engl J Med. 373(1):23-34 (2015)) or nivolumab 3 mg/kg+IPI 3 mg/kg (55% and 54% for CheckMate 067 and CheckMate 069, respectively) (Id., Hodi et al. Combined nivolumab and ipilimumab versus ipilimumab alone in patients with advanced melanoma: 2-year overall survival outcomes in a multicenter, randomized, controlled, phase 2 trial. Lancet Oncol. 17(11):1558-1568 (2016)). 72% of the subjects were able to complete all 4 planned doses of IPI, however, DRAEs were present in 42% of the subjects (Long et al. Presented at 2016 ASCO Annual Meeting Jun. 3-7, 2016; Chicago, IL; abstract 9506).

### EXAMPLE 2

### A Phase I/II Randomized Trial of Pembrolizumab Plus 2 Fixed-Dose Regimens of Ipilimumab for Advanced Melanoma

### Objectives

The primary objectives of this study are to establish the safety and tolerability of a 200 mg fixed dose of pembrolizumab in combination with 2 schedules of reduced fixed dose ipilimumab and to evaluate overall response rate (ORR) per RECIST v1.1 by independent central review of 200 mg fixed dose of pembrolizumab in combination with 2 schedules of reduced fixed dose ipilimumab.

The secondary objectives of this study are to: (1) evaluate the efficacy of the combination regimens with respect to ordinal response score (defined as best ORR calculated as complete response (CR), very good partial response (VGPR); defined as >60% change from baseline in tumor size per RECIST v1.1, moderate partial response (PR) (defined as >30% to ≤60% change from baseline tumor size), stable disease (SD), or progressive disease (PD) based on the degree of tumor shrinkage) per RECIST v1.1 by independent central review; (2) to evaluate duration of response (DOR) and progression free survival (PFS) of the combination regimens per RECIST v1.1 by independent central review; (3) to evaluate the overall survival (OS) of the combination regimens; and (4) to evaluate the relationship between PD-L1 expression and ORR, PFS, and OS in patients treated with the combination regimens.

Additional exploratory objectives of this study are to: (1) evaluate ORR of the combination regimens per RECIST v1.1 by central review using tumor length and tumor volumetric changes; (2) evaluate ORR per RECIST v1.1 by independent central review in patients who stopped pembrolizumab with SD or better, and who were retreated with combination therapy or pembrolizumab monotherapy following subsequent disease progression; (3) investigate the relationship between potential biomarkers and anti-tumor activity of pembrolizumab; (4) investigate the rate of pembrolizumab anti-drug antibodies and impact on pharmacokinetics; and (5) correlate use of time to growth modeling and simulation with clinical outcomes in patients treated with the combination regimens.

### Study Design

Approximately 100 patients will be randomly assigned 1:1 to receive either: (1) Pembrolizumab 200 mg Q3W plus ipilimumab 50 mg Q6W for ≤4 doses (arm 1), or (2) Pembrolizumab 200 mg Q3W plus ipilimumab 100 mg Q12W for ≤4 doses (arm 2). Combination therapy will continue for ≤24 weeks in arm 1 or ≤48 weeks in arm 2, followed by pembrolizumab monotherapy for ≤24 months or until documented PD, unacceptable toxicity, patient withdrawal of consent, or investigator decision

Patients with investigator-determined confirmed CR per modified RECIST v1.1 who have been treated with pembrolizumab for ≥24 weeks and who have received ≥2 doses of pembrolizumab beyond initial determination of CR could discontinue pembrolizumab; patients with investigator-determined confirmed CR or VGPR who received ≥1 ipilimumab dose could discontinue ipilimumab.

Patients experiencing SD or better per modified RECIST v1.1 who subsequently experience PD may be eligible for retreatment with pembrolizumab plus ipilimumab or with pembrolizumab monotherapy for a maximum of 17 doses of pembrolizumab and 4 doses of ipilimumab.

Eligible patients who experience radiologic PD per modified RECIST v1.1 may remain on treatment until a confirmatory scan 24 weeks later. Clinically stable patients with confirmed PD per modified RECIST v1.1 who are deriving clinical benefit may continue treatment at the discretion of the investigator.

### Assessments and Follow-Up

Response will be assessed by tumor imaging every 6 weeks until week 24, then every 12 weeks thereafter per RECIST v1.1 by independent central review (for efficacy) and per modified RECIST v1.1 by investigator review (for eligibility and treatment decisions). A 5-category ordinal response score per RECIST v1.1 by independent central review will be used to assess best overall response calculated as CR, VGPR, moderate PR, SD, or PD. Adverse events (AEs) will be assessed throughout treatment and for 30 days thereafter (90 days for serious AEs) and will be graded per National Cancer Institute Common Terminology Criteria for Adverse Events, version 4.0. After confirmed PD or start of new anticancer therapy, patients will be contacted by telephone every 12 weeks to monitor survival.

### Analyses

For the analysis of efficacy, all patients with measurable disease at baseline will serve as the primary efficacy population. Exact 90% confidence intervals for ORR will be calculated for the true ORR. Descriptive statistics will be used to assess ordinal response score. The Kaplan-Meier method will be used to estimate PFS, OS and DOR Only patients who achieve a CR or PR will be included in the DOR analysis

For the analysis of safety, all randomly assigned patients who receive ≥1 dose of study treatment will serve as the primary safety population. Enrollment is currently ongoing.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

TABLE 5
provides a brief description of the PD-1-related
sequences in the sequence listing.

| SEQ ID | |
|---|---|
| NO: | Description |
| 1 | hPD-1.08A light chain CDR1 |
| 2 | hPD-1.08A light chain CDR2 |
| 3 | hPD-1-08A light chain CDR3 |
| 4 | hPD-1.08A heavy chain CDR1 |
| 5 | hPD-1.08A heavy chain CDR2 |
| 6 | hPD-1.08A heavy chain CDR3 |
| 7 | hPD-1.09A light chain CDR1 |
| 8 | hPD-1.09A light chain CDR2 |
| 9 | hPD-1.09A light chain CDR3 |
| 10 | hPD-1.09A heavy chain CDR1 |
| 11 | hPD-1.09A heavy chain CDR2 |
| 12 | hPD-1.09A heavy chain CDR3 |
| 13 | 109A-H heavy chain variable |
| 14 | 409A-H heavy chain full length |
| 15 | K09A-L-11 light chain variable |
| 16 | K09A-L-16 light chain variable |
| 17 | K09A-L-17 light chain variable |
| 18 | K09A-L-11 light chain full length |
| 19 | K09A-L-16 light chain full length |
| 20 | K09A-L-17 light chain full length |
| 21 | Pembrolizumab heavy chain |
| 22 | Pembrolizumab light chain |
| 23 | Nivolumab heavy chain |
| 24 | Nivolumab light chain |
| 25 | Precursor human PD-L1 |
| 26 | Nivolumab heavy chain variable |
| 27 | Nivolumab light chain variable |
| 28 | Nivolumab heavy chain CDR1 |
| 29 | Nivolumab heavy chain CDR2 |
| 30 | Nivolumab heavy chain CDR3 |
| 31 | Nivolumab light chain CDR1 |
| 32 | Nivolumab light chain CDR2 |
| 33 | Nivolumab light chain CDR3 |

TABLE 6
provides a brief description of the CTLA4-related
sequences in the sequence listing.

| SEQ ID | |
|---|---|
| NO: | Description |
| 34 | CTLA4 Ab heavy chain CDR1 |
| 35 | CTLA4 Ab heavy chain CDR2 |
| 36 | CTLA4 Ab heavy chain CDR3 |
| 37 | CTLA4 Ab light chain CDR1 |
| 38 | CTLA4 Ab light chain CDR2 |
| 39 | CTLA4 Ab light chain CDR3 |
| 40 | CTLA4 Ab light chain CDR3 |
| 41 | CTLA4 Ab light chain CDR3 |
| 42 | 8D2/8D2 (RE) heavy chain |
| | variable |
| 43 | 8D2/8D2 (RE) light chain |
| | variable |
| 44 | 8D2H1L1 heavy chain variable |
| 45 | 8D2H1L1 light chain variable |
| 46 | 8D2H2L2 heavy chain variable |
| 47 | 8D2H2L2 light chain variable |
| 48 | 8D3H3L3 heavy chain variable |
| 49 | 8D3H3L3 light chain variable |
| 50 | 8D2H2L15 heavy chain variable |
| 51 | 8D2H2L15 light chain variable |
| 52 | 8D2H2l17 heavy chain variable |
| 53 | 8D2H2l17 light chain variable |
| 54 | murine 4G10 heavy chain |
| | variable |
| 55 | murine 4G10 light chain variable |
| 56 | CTLA4 Ab heavy chain CDR1 |
| 57 | CTLA4 Ab heavy chain CDR1 |
| 58 | CTLA4 Ab heavy chain CDR2 |
| 59 | CTLA4 Ab heavy chain CDR2 |
| 60 | CTLA4 Ab heavy chain CDR3 |
| 61 | CTLA4 Ab heavy chain CDR3 |
| 62 | CTLA4 Ab light chain CDR1 |
| 63 | CTLA4 Ab light chain CDR1 |
| 64 | CTLA4 Ab light chain CDR2 |
| 65 | CTLA4 Ab light chain CDR3 |
| 66 | CTLA4 Ab light chain CDR3 |
| 67 | 4G10H1 humanized, heavy chain |
| | variable |
| 68 | 4G10H3 humanized, heavy chain |
| | variable |
| 69 | 4G10H4 humanized, heavy chain |
| | variable |
| 70 | 4G10H5 humanized, heavy chain |
| | variable |
| 71 | 4G10H consensus humanized, |
| | heavy chain variable |
| 72 | 4G10L1 humanized, light chain |
| | variable |
| 73 | 4G10L3 humanized, light chain |
| | variable |
| 74 | 4G10Lconsensus humanized, |
| | light chain variable |
| 75 | 4G10H1L1 heavy chain variable |
| 76 | 4G10H1L1 light chain variable |
| 77 | 4G10H3L3 heavy chain variable |
| 78 | 4G10H3L3 light chain variable |
| 79 | 4G10H4L3 heavy chain variable |
| 80 | 4G10H4L3 light chain variable |
| 81 | 4G10H5L3 heavy chain variable |
| 82 | 4G10H5L3 light chain variable |
| 83 | Ipilimumab heavy chain |
| 84 | Ipilimumab light chain |
| 85 | Ipilimumab HCDR1 |
| 86 | Ipilimumab HCDR2 |
| 87 | Ipilimumab HCDR3 |
| 88 | Ipilimumab LCDR1 |
| 89 | Ipilimumab LCDR2 |
| 90 | Ipilimumab LCDR3 |
| 91 | Ipilimumab heavy chain variable |
| 92 | Ipilimumab light chain variable |
| 93 | 8D2/8D2 (RE) VH consensus |
| 94 | 8D2/8D2 (RE) VH Variant 1 |
| 95 | 8D2H1L1 VH consensus |
| 96 | 8D2H1L1 VH Variant 1 |
| 97 | 8D2H2L2 VH consensus |
| 98 | 8D2H2L2 VH Variant 1 |
| 99 | 8D2H1L15 VH consensus |
| 100 | 8D2H1L15 VH Variant 1 |
| 101 | 8D2H1L17 VH consensus |
| 102 | 8D2H1L17 VH Variant 1 |

1-40. (canceled)

41. A method of treating cancer in a human patient comprising administering:
(a) 200 mg of an anti-PD-1 antibody or antigen binding fragment thereof and 25 mg or 50 mg of an anti-CTLA4 antibody or antigen binding fragment thereof; or
(b) 2 mg/kg of an anti-PD-1 antibody or antigen binding fragment thereof and 25 mg or 50 mg of an anti-CTLA4 antibody or antigen binding fragment thereof,
wherein the anti-PD-1 antibody or antigen binding fragment thereof is administered once every three weeks and the anti-CTLA4 antibody or antigen binding fragment thereof is administered once every three weeks or once every six weeks,
wherein the anti-PD-1 antibody or antigen binding fragment comprises: light chain complementarity determining regions (CDRs) comprising amino acid sequences as set forth in SEQ ID Nos: 7, 8, and 9 and heavy chain CDRs comprising amino acid sequences as set forth in SEQ ID Nos: 10, 11, and 12, and
wherein the anti-CTLA4 antibody or antigen binding fragment comprises: light chain complementarity determining regions (CDRs) comprising amino acid sequences as set forth in SEQ ID Nos: 37, 38, and 39 and heavy chain CDRs comprising amino acid sequences as set forth in SEQ ID Nos: 34, 35, and 36.

42. The method of claim 41, comprising administering 200 mg of an anti-PD-1 antibody and 50 mg of an anti-CTLA4 antibody.

43. The method of claim 41, comprising administering 200 mg of an anti-PD-1 antibody and 25 mg of an anti-CTLA4 antibody.

44. The method of claim 41, comprising administering 2 mg/kg of an anti-PD-1 and 50 mg of an anti-CTLA4 antibody.

45. The method of claim 41, comprising administering 2 mg/kg of an anti-PD-1 antibody and 25 mg of an anti-CTLA4 antibody.

46. The method of claim 41, further comprising continuing the administration of the anti-PD-1 antibody once every three weeks after administration of the anti-CTLA4 antibody is discontinued.

47. The method of claim 41, further comprising continuing the administration of the anti-PD-1 antibody once every three weeks after administration of the anti-CTLA4 antibody is discontinued.

48. The method of claim 41, wherein the cancer is melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

49. The method of claim 41, wherein the anti-PD-1 antibody is pembrolizumab.

50. The method of claim 41, wherein the anti-CTLA4 antibody is 8D2H2L2, or variant thereof, wherein the methionine at position 18 in the variable heavy (VH) chain amino acid sequence of the 8D2H2L2 variant is substituted with an amino acid selected from: leucine, valine, isoleucine, and alanine.

51. The method of claim 41, wherein the anti-CTLA4 antibody comprises a heavy chain variable region comprising the amino acid sequence as set forth in SEQ ID NO: 97, wherein the X at position 18 of the amino acid sequence is leucine, and a light chain variable region comprising the amino acid sequence as set forth in SEQ ID NO: 47.

52. A composition comprising a dosage of an anti-PD-1 antibody or an antigen binding fragment thereof and a dosage of an anti-CTLA4 antibody or an antigen binding fragment thereof, wherein the dosage is selected from the group consisting of:
(i) 200 mg of an anti-PD-1 antibody or an antigen binding fragment thereof and 25 mg of an anti-CTLA4 antibody or an antigen binding fragment thereof,
(ii) 200 mg of an anti-PD-1 antibody or an antigen binding fragment thereof and 50 mg of an anti-CTLA4 antibody or an antigen binding fragment thereof,
(iii) 2 mg/kg of an anti-PD-1 antibody or an antigen binding fragment thereof and 25 mg of an anti-CTLA4 antibody or an antigen binding fragment thereof,
(iv) 2 mg/kg of an anti-PD-1 antibody or an antigen binding fragment thereof and 50 mg of an anti-CTLA4 antibody or an antigen binding fragment thereof, and
wherein the anti-PD-1 antibody or antigen binding fragment comprises light chain complementarity determining regions (CDRs) comprising amino acid sequences as set forth in SEQ ID Nos: 7, 8, and 9 and heavy chain CDRs comprising amino acid sequences as set forth in SEQ ID Nos: 10, 11, and 12 and
wherein the anti-CTLA4 antibody or antigen binding fragment comprises: light chain complementarity determining regions (CDRs) comprising amino acid sequences as set forth in SEQ ID Nos: 37, 38, and 39 and heavy chain CDRs comprising amino acid sequences as set forth in SEQ ID Nos: 34, 35, and 36.

53. The composition of claim 52, wherein the anti-PD-1 antibody is pembrolizumab and the anti-CTLA4 antibody is 8D2H2L2 or variant thereof, wherein the methionine at position 18 in the variable heavy (VH) chain amino acid sequence of the anti-CTLA4 antibody is substituted with leucine.

54. The composition of any claim 52 for treating an individual with cancer, wherein the cancer is melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, or salivary cancer.

55. A kit for treating a patient with cancer, the kit comprising:
(a) a dosage of an anti-PD-1 antibody or antigen binding fragment thereof and a dosage of an anti-CTLA4 antibody or antigen binding fragment thereof selected from the group consisting of:
(i) 200 mg of an anti-PD-1 antibody or antigen binding fragment thereof and 25 mg of an anti-CTLA4 antibody or antigen binding fragment thereof; or
(ii) 200 mg of an anti-PD-1 antibody or antigen binding fragment thereof and 50 mg of an anti-CTLA4 antibody or antigen binding fragment thereof; and
(b) instructions for using the anti-PD-1 antibody or antigen binding fragment thereof and the anti-CTLA4 antibody or antigen binding fragment thereof;
wherein the anti-PD-1 antibody or antigen binding fragment comprises: light chain complementarity determining regions (CDRs) comprising amino acid sequences as set forth in SEQ ID Nos: 7, 8, and 9 and heavy chain CDRs comprising amino acid sequences as set forth in SEQ ID Nos: 10, 11, and 12, and
wherein the anti-CTLA4 antibody or antigen binding fragment comprises: light chain complementarity determining regions (CDRs) comprising amino acid sequences as set forth in SEQ ID Nos: 37, 38, and 39 and heavy chain CDRs comprising amino acid sequences as set forth in SEQ ID Nos: 34, 35, and 36.

56. The kit of claim 55, wherein the anti-PD-1 antibody is pembrolizumab and the anti-CTLA4 antibody is 8D2H2L2 or variant thereof, wherein the methionine at position 18 in the variable heavy (VH) chain amino acid sequence of the anti-CTLA4 antibody is substituted with leucine.


SEQUENCE LISTING (106 sequences)

SEQ ID NO: 1 - protein, 15 aa, synthetic construct
  REGION 1..15: note = hPD-1.08A LC CDR1
RASKSVSTSGFSYLH

SEQ ID NO: 2 - protein, 7 aa, synthetic construct
  REGION 1..7: note = hPD-1.08A LC CDR2
LASNLES

SEQ ID NO: 3 - protein, 9 aa, synthetic construct
  REGION 1..9: note = hPD-1.08A LC CDR3
QHSWELPLT

SEQ ID NO: 4 - protein, 5 aa, synthetic construct
  REGION 1..5: note = hPD-1.08 HC CDR1
SYYLY

SEQ ID NO: 5 - protein, 17 aa, synthetic construct
  REGION 1..17: note = hPD-1.08 HC CDR2
GVNPSNGGTNFSEKFKS

SEQ ID NO: 6 - protein, 11 aa, synthetic construct
  REGION 1..11: note = hPD-1.08 HC CDR3
RDSNYDGGFDY

SEQ ID NO: 7 - protein, 15 aa, synthetic construct
  REGION 1..15: note = hPD-1.09A LC CDR1
RASKGVSTSGYSYLH

SEQ ID NO: 8 - protein, 7 aa, synthetic construct
  REGION 1..7: note = hPD-1.09A LC CDR2
LASYLES

SEQ ID NO: 9 - protein, 9 aa, synthetic construct
  REGION 1..9: note = hPD-1.09A LC CDR3
QHSRDLPLT

SEQ ID NO: 10 - protein, 5 aa, synthetic construct
  REGION 1..5: note = hPD-1.09A HC CDR 1
NYYMY

SEQ ID NO: 11 - protein, 17 aa, synthetic construct
  REGION 1..17: note = hPD-1.09 A HC CDR 2
GINPSNGGTNFNEKFKN

SEQ ID NO: 12 - protein, 11 aa, synthetic construct
  REGION 1..11: note = hPD 1.09A HC CDR 3
RDYRFDMGFDY

SEQ ID NO: 13 - protein, 120 aa, synthetic construct [sequence omitted]

SEQ ID NO: 14 - protein, 447 aa, synthetic construct
  REGION 1..447: note = 409A-HC full length
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 111 aa, synthetic construct [sequence omitted]

SEQ ID NO: 16 - protein, 111 aa, synthetic construct
  REGION 1..111: note = k09A-L-16 LC variable
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 17 - protein, 111 aa, synthetic construct
  REGION 1..111: note = K09A-L-17 LC variable
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 18 - protein, 218 aa, synthetic construct
  REGION 1..218: note = K09A-L-11- LC full length
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 218 aa, synthetic construct
  REGION 1..218: note = K09A-L-16 LC full length
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 218 aa, synthetic construct
  REGION 1..218: note = K09A-L-17 LC full length
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct; identical to SEQ ID NO: 14
  REGION 1..447: note = heavy chain

SEQ ID NO: 22 - protein, 218 aa, synthetic construct; identical to SEQ ID NO: 18
  REGION 1..218: note = light chain

SEQ ID NO: 23 - protein, 440 aa, synthetic construct
  REGION 1..440: note = heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, synthetic construct
  REGION 1..214: note = light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 290 aa, synthetic construct
  REGION 1..290: note = Precursor human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 26 - protein, 113 aa, synthetic construct [sequence omitted]

SEQ ID NO: 27 - protein, 107 aa, synthetic construct [sequence omitted]

SEQ ID NO: 28 - protein, 5 aa, synthetic construct
  REGION 1..5: note = heavy chain CDR1
NSGMH

SEQ ID NO: 29 - protein, 17 aa, synthetic construct
  REGION 1..17: note = heavy chain CDR 2
VIWYDGSKRYYADSVKG

SEQ ID NO: 30 - protein, 4 aa, synthetic construct
  REGION 1..4: note = heavy chain CDR 3
NDDY

SEQ ID NO: 31 - protein, 11 aa, synthetic construct
  REGION 1..11: note = light chain CDR 1
RASQSVSSYLA

SEQ ID NO: 32 - protein, 7 aa, synthetic construct
  REGION 1..7: note = light chain CDR 2
DASNRAT

SEQ ID NO: 33 - protein, 9 aa, synthetic construct
  REGION 1..9: note = light chain CDR 3
QQSSNWPRT

SEQ ID NO: 34 - protein, 8 aa, synthetic construct
  REGION 1..8: note = CDR
GFTFSDNW

SEQ ID NO: 35 - protein, 10 aa, synthetic construct
  REGION 1..10: note = CDR
IRNKPYNYET

SEQ ID NO: 36 - protein, 6 aa, synthetic construct
  REGION 1..6: note = CDR
TAQFAY

SEQ ID NO: 37 - protein, 6 aa, synthetic construct
  REGION 1..6: note = CDR
ENIYGG

SEQ ID NO: 38 - skipped

SEQ ID NO: 39 - protein, 9 aa, synthetic construct
  REGION 1..9: note = CDR
QNVLRSPFT

SEQ ID NO: 40 - protein, 9 aa, synthetic construct
  REGION 1..9: note = CDR
QNVLSRHPG

SEQ ID NO: 41 - protein, 9 aa, synthetic construct
  REGION 1..9: note = CDR
QNVLSSRPG

SEQ ID NO: 42 - protein, 115 aa, synthetic construct
  REGION 1..115: note = heavy chain variable
EVKLDETGGGLVQPGRPMKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 43 - protein, 106 aa, synthetic construct
  REGION 1..106: note = light chain variable
DIQMTQSPASLSASVGETVTITCGTSENIYGGLNWYQRKQGKSPQLLIFGATNLADGMSSRFSGSGSGRQYSLKISSLHPDDVATYYCQNVLRSPFTFGSGTKLEI

SEQ ID NO: 44 - protein, 115 aa, synthetic construct
  REGION 1..115: note = heavy chain variable
EVQLVESGGGLVQPGGSMRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 45 - protein, 107 aa, synthetic construct
  REGION 1..107: note = light chain variable
DIQMTQSPSSLSASVGDRVTITCRTSENIYGGLNWYQRKQGKSPKLLIYGATNLASGMSSRFSGSGSGTDYTLKISSLHPDDVATYYCQNVLRSPFTFGSGTKLEIK

SEQ ID NO: 46 - protein, 115 aa, synthetic construct
  REGION 1..115: note = heavy chain variable
EVQLVESGGGLVQPGGSMRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 47 - protein, 107 aa, synthetic construct
  REGION 1..107: note = light chain variable
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIK

SEQ ID NO: 48 - protein, 115 aa, synthetic construct
  REGION 1..115: note = heavy chain variable
EVQLVESGGGLVQPGGSLRLSCAASGFTFSDNWMNWVRQAPGKGLEWVAQIRNKPYNYETEYAASVKGRFTISRDDSKNSAYLQMNSLKTEDTAVYYCTAQFAYWGQGTLVTVSS

SEQ ID NO: 49 - protein, 107 aa, synthetic construct
  REGION 1..107: note = light chain variable
DIQMTQSPSSLSASVGDRVTITCRASENIYGGLNWYQQKPGKAPKLLIYGATSLASGVPSRFSGSGSGTDYTLTISSLQPEDFATYYCQNVLRSPFTFGSGTKLEIK

SEQ ID NO: 50 - protein, 115 aa, synthetic construct; identical to SEQ ID NO: 46
  REGION 1..115: note = heavy chain variable

SEQ ID NO: 51 - protein, 107 aa, synthetic construct
  REGION 1..107: note = light chain variable
DIQMTQSPSSLSASVGDRVTITCRTSENIYGGLNWYQRKPGKSPKLLIYGATNLASGVSSRFSGSGSGTDYTLTISSLQPEDVATYYCQNVLSRHPGFGSGTKLEIK

SEQ ID NO: 52 - protein, 115 aa, synthetic construct; identical to SEQ ID NO: 46
  REGION 1..115: note = heavy chain variable

SEQ ID NO: 53 - protein, 107 aa, synthetic construct
  REGION 1..107: note = light chain variable
DIQMTQSPSSLSASVGDRVTITCRTSENIYGGLNWYQRKPGKSPKLLIYGATNLASGVSSRFSGSGSGTDYTLTISSLQPEDVATYYCQNVLSSRPGFGSGTKLEIK

SEQ ID NO: 54 - protein, 115 aa, synthetic construct
  REGION 1..115: note = murine heavy chain variable
QVKLQESGPELVKPGASMKISCKASGYSFTGYTMNWVKQSHGKNLEWIGLINPYNNITNYNQKFMGKATFTVDKSSSTAYMELLRLTSEDSGVYFCARLDYRSYWGQGTLVTVSA

SEQ ID NO: 55 - protein, 126 aa, synthetic construct
  REGION 1..126: note = murine light chain variable
QAVVTQESALTTSPGETVTLTCRSSTGAVTTSNFANWVQEKPDHLFTSLIGGTNNRAPGVPARFSGSLIGDKAALTITGAQTEDEAIYFCALWYSNHWVFGGGTKLTVLGQPKSSPSVTLFQGQFC

SEQ ID NO: 56 - protein, 8 aa, synthetic construct
  REGION 1..8: note = CDR
GYSFTGYT

SEQ ID NO: 57 - protein, 5 aa, synthetic construct
  REGION 1..5: note = CDR
  VARIANT 4: note = MISC_FEATURE - X = Met, Val, Leu, Ile, Gly, Ala, Ser, or Thr
GYTXN

SEQ ID NO: 58 - protein, 8 aa, synthetic construct
  REGION 1..8: note = CDR
  VARIANT 6: note = MISC_FEATURE - X = Asn, Asp, or Glu
  VARIANT 8: note = MISC_FEATURE - X = Thr, Asp, Glu, Gly, or Ala
INPYNXIX

SEQ ID NO: 59 - protein, 17 aa, synthetic construct
  REGION 1..17: note = CDR
  VARIANT 7: note = MISC_FEATURE - X = Asn or Asp
  VARIANT 9: note = MISC_FEATURE - X = Thr, Asp, Glu, Gly, or Ala
  VARIANT 12: note = MISC_FEATURE - X = Ala or Asn
  VARIANT 16: note = MISC_FEATURE - X = Gln or Met
LINPYNXIXNYXQKFXG

SEQ ID NO: 60 - protein, 6 aa, synthetic construct
  REGION 1..6: note = CDR
LDYRSY

SEQ ID NO: 61 - protein, 8 aa, synthetic construct
  REGION 1..8: note = CDR
ARLDYRSY

SEQ ID NO: 62 - protein, 9 aa, synthetic construct
  REGION 1..9: note = CDR
TGAVTTSNF

SEQ ID NO: 63 - protein, 14 aa, synthetic construct
  REGION 1..14: note = CDR
  VARIANT 13: note = MISC_FEATURE - X = Pro or Ala
GSSTGAVTTSNFXN

SEQ ID NO: 64 - protein, 7 aa, synthetic construct
  REGION 1..7: note = CDR
  VARIANT 5: note = MISC_FEATURE - X = Lys or Arg
  VARIANT 7: note = MISC_FEATURE - X = any amino acid EXCEPT Met or Cys
  VARIANT 7: note = MISC_FEATURE - X = Ser or Pro
GTNNXAX

SEQ ID NO: 65 - protein, 8 aa, synthetic construct
  REGION 1..8: note = CDR
  VARIANT 3: note = MISC_FEATURE - X = Trp or any amino acid except Met or Cys
  VARIANT 8: note = MISC_FEATURE - X = Trp or any amino acid except Met or Cys
ALXYSNHX

SEQ ID NO: 66 - protein, 9 aa, synthetic construct
  REGION 1..9: note = CDR
  VARIANT 3: note = MISC_FEATURE - X = Trp or any amino acid EXCEPT Met or Cys
  VARIANT 8: note = MISC_FEATURE - X = Trp or any amino acid EXCEPT Met or Cys
ALXYSNHXV

SEQ ID NO: 67 - protein, 115 aa, synthetic construct
  REGION 1..115: note = heavy chain variable
QVQLVESGAELVKPGASMKISCKASGYSFTGYTMNWVKQAPGQGLEWIGLINPYNNITNYNQKFMGKATFTVDKSISTAYMELSRLTSDDSGVYFCARLDYRSYWGQGTLVTVSA

SEQ ID NO: 68 - protein, 115 aa, synthetic construct
  REGION 1..115: note = heavy chain variable
QVQLVESGAEVKKPGASVKVSCKASGYSFTGYTMNWVRQAPGQGLEWIGLINPYNNITNYAQKFQGRVTFTVDTSISTAYMELSRLRSDDTGVYFCARLDYRSYWGQGTLVTVSA

SEQ ID NO: 69 - protein, 115 aa, synthetic construct
  REGION 1..115: note = heavy chain variable
QVQLVESGAEVKKPGASVKVSCKASGYSFTGYTMNWVRQAPGQGLEWIGLINPYNDITNYAQKFQGRVTFTVDTSISTAYMELSRLRSDDTGVYFCARLDYRSYWGQGTLVTVSA

SEQ ID NO: 70 - protein, 115 aa, synthetic construct
  REGION 1..115: note = heavy chain variable
QVQLVESGAEVKKPGASVKVSCKASGYSFTGYTMNWVRQAPGQGLEWIGLINPYNNIDNYAQKFQGRVTFTVDTSISTAYMELSRLRSDDTGVYFCARLDYRSYWGQGTLVTVSA

SEQ ID NO: 71 - protein, 115 aa, synthetic construct
  REGION 1..115: note = heavy chain variable
  VARIANT 11: note = MISC_FEATURE - X = Val or Leu
  VARIANT 18: note = MISC_FEATURE - X = Val or Met
  VARIANT 20: note = MISC_FEATURE - X = Val or Ile
  VARIANT 34: note = MISC_FEATURE - X = Met, Val, Leu, Ile, Gly, Ala, Ser or Thr
  VARIANT 38: note = MISC_FEATURE - X = Arg or Lys
  VARIANT 56: note = MISC_FEATURE - X = Asn, Asp, or Glu
  VARIANT 58: note = MISC_FEATURE - X = Thr, Asp, Glu, Gly, or Ala
  VARIANT 61: note = MISC_FEATURE - X = Ala or Asn
  VARIANT 65: note = MISC_FEATURE - X = Gln or Met
  VARIANT 67: note = MISC_FEATURE - X = Gln or Met
  VARIANT 68: note = MISC_FEATURE - X = Val or Ala
  VARIANT 74: note = MISC_FEATURE - X =Thr or Lys
  VARIANT 87: note = MISC_FEATURE - X =Arg or Thr
  VARIANT 91: note = MISC_FEATURE - X = The or Ser
QVQLVESGAEXKKPGASXKXSCKASGYSFTGYTXNWVXQAPGQGLEWIGLINPYNXIXNYXQKFXGXXTFTVDXSISTAYMELSRLXSDDXGVYFCARLDYRSYWGQGTLVTVSA

SEQ ID NO: 72 - protein, 109 aa, synthetic construct
  REGION 1..109: note = light chain variable
QAVVTQEPSLTVSPGGTVTLTCGSSTGAVTTSNFANWVQEKPGQAFRSLIGGTNNRASWVPARFSGSLLGGKAALTISGAQPEDEAEYFCALWYSNHWVFGGGTKLTVL

SEQ ID NO: 73 - protein, 109 aa, synthetic construct
  REGION 1..109: note = light chain variable
QAVVTQEPSLTVSPGGTVTLTCGSSTGAVTTSNFPNWVQQKPGQAPRSLIGGTNNKASWTPARFSGSLLGGKAALTISGAQPEDEAEYYCALWYSNHWVFGGGTKLTVL

SEQ ID NO: 74 - protein, 109 aa, synthetic construct
  REGION 1..109: note = light chain variable
  VARIANT 35: note = MISC_FEATURE - X = Pro or Ala
  VARIANT 40: note = MISC_FEATURE - X = Gln or Glu
  VARIANT 46: note = MISC_FEATURE - X = Pro of Phe
  VARIANT 56: note = MISC_FEATURE - X =Lys, Arg or any amino acid EXCEPT Met or Cys
  VARIANT 58: note = MISC_FEATURE - X =Ser or Pro
  VARIANT 60: note = ISC_FEATURE - X =Thr or Val
  VARIANT 89: note = MISC_FEATURE - X =Tyr or Phe
  VARIANT 93: note = MISC_FEATURE - X = Trp or any amino acid except Met or Cys
  VARIANT 98: note = MISC_FEATURE - X = Trp or any amino acid except Met or Cys
QAVVTQEPSLTVSPGGTVTLTCGSSTGAVTTSNFXNWVQXKPGQAXRSLIGGTNNXAXWXPARFSGSLLGGKAALTISGAQPEDEAEYXCALXYSNHXVFGGGTKLTVL

SEQ ID NO: 75 - protein, 115 aa, synthetic construct; identical to SEQ ID NO: 67
  REGION 1..115: note = heavy chain variable

SEQ ID NO: 76 - protein, 109 aa, synthetic construct; identical to SEQ ID NO: 72
  REGION 1..109: note = light chain variable

SEQ ID NO: 77 - protein, 115 aa, synthetic construct; identical to SEQ ID NO: 68
  REGION 1..115: note = heavy chain variable

SEQ ID NO: 78 - protein, 109 aa, synthetic construct; identical to SEQ ID NO: 73
  REGION 1..109: note = light chain variable

SEQ ID NO: 79 - protein, 115 aa, synthetic construct; identical to SEQ ID NO: 69
  REGION 1..115: note = heavy chain variable

SEQ ID NO: 80 - protein, 109 aa, synthetic construct; identical to SEQ ID NO: 73
  REGION 1..109: note = light chain variable

SEQ ID NO: 81 - protein, 115 aa, synthetic construct; identical to SEQ ID NO: 70
  REGION 1..115: note = heavy chain variable

SEQ ID NO: 82 - protein, 109 aa, synthetic construct; identical to SEQ ID NO: 73
  REGION 1..109: note = light chain variable

SEQ ID NO: 83 - protein, 448 aa, synthetic construct
  REGION 1..448: note = heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 215 aa, synthetic construct
  REGION 1..215: note = light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 5 aa, synthetic construct
  REGION 1..5: note = CDR
SYTMH

SEQ ID NO: 86 - protein, 17 aa, synthetic construct
  REGION 1..17: note = CDR
FISYDGNNKYYADSVKG

SEQ ID NO: 87 - protein, 9 aa, synthetic construct
  REGION 1..9: note = CDR
TGWLGPFDY

SEQ ID NO: 88 - protein, 12 aa, synthetic construct
  REGION 1..12: note = CDR
RASQSVGSSYLA

SEQ ID NO: 89 - protein, 7 aa, synthetic construct
  REGION 1..7: note = CDR
GAFSRAT

SEQ ID NO: 90 - protein, 9 aa, synthetic construct
  REGION 1..9: note = CDR
QQYGSSPWT

SEQ ID NO: 91 - protein, 118 aa, synthetic construct
  REGION 1..118: note = heavy chain variable
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 92 - protein, 108 aa, synthetic construct
  REGION 1..108: note = light chain variable
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 93 - protein, 115 aa, synthetic construct
  REGION 1..115: note = variable heavy chain
  SITE 18: note = MISC_FEATURE - X = Leu, Val, Ile or Ala
EVKLDETGGGLVQPGRPXKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 94 - protein, 115 aa, synthetic construct
  REGION 1..115: note = variable heavy chain
EVKLDETGGGLVQPGRPIKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 95 - protein, 115 aa, synthetic construct
  REGION 1..115: note = variable heavy chain
  VARIANT 18: note = MISC_FEATURE - X = Leu, Val, Ile or Ala
EVQLVESGGGLVQPGGSXRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 96 - protein, 115 aa, synthetic construct
  REGION 1..115: note = variable heavy chain
EVQLVESGGGLVQPGGSIRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 97 - protein, 115 aa, synthetic construct
  REGION 1..115: note = variable heavy chain
  VARIANT 18: note = MISC_FEATURE - X = Leu, Val, Ile or Ala
EVQLVESGGGLVQPGGSXRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 98 - protein, 115 aa, synthetic construct
  REGION 1..115: note = variable heavy chain
EVQLVESGGGLVQPGGSIRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 99 - protein, 115 aa, synthetic construct; identical to SEQ ID NO: 97
  REGION 1..115: note = variable heavy chain
  VARIANT 18: note = MISC_FEATURE - X = Leu, Val, Ile or Ala

SEQ ID NO: 100 - protein, 115 aa, synthetic construct; identical to SEQ ID NO: 98
  REGION 1..115: note = variable heavy chain

SEQ ID NO: 101 - protein, 115 aa, synthetic construct; identical to SEQ ID NO: 97
  REGION 1..115: note = variable heavy chain
  SITE 18: note = MISC_FEATURE - X = Leu, Val, Ile or Ala

SEQ ID NO: 102 - protein, 115 aa, synthetic construct; identical to SEQ ID NO: 98
  REGION 1..115: note = variable heavy chain

SEQ ID NO: 103 - protein, 115 aa, synthetic construct; identical to SEQ ID NO: 98
  REGION 1..115: note = light chain variable

SEQ ID NO: 104 - protein, 122 aa, synthetic construct
  REGION 1..122: note = heavy chain variable
QVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSS

SEQ ID NO: 105 - protein, 112 aa, synthetic construct
  REGION 1..112: note = light chain variable
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 106 - protein, 122 aa, synthetic construct
  REGION 1..122: note = heavy chain variable
QVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS